(19) United States
(12) Patent Application Publication
(10) Pub. No.: US 2014/0308699 A1
(43) Pub. Date: Oct. 16, 2014

(54) YEAST ARTIFICIAL CHROMOSOME CARRYING THE MAMMALIAN GLYCOSYLATION PATHWAY

(71) Applicants: Christophe Javaud, Uzerche (FR); Vincent Carre, Jabreilles-Les-Bordes (FR)

(72) Inventors: Christophe Javaud, Uzerche (FR); Vincent Carre, Jabreilles-Les-Bordes (FR)

(73) Assignee: GLYCODE, Uzerche (FR)

(21) Appl. No.: 13/812,473

(22) PCT Filed: Aug. 1, 2011
(86) PCT No.: PCT/EP2011/063247

§ 371 Date: Sep. 6, 2013

Related U.S. Application Data
(60) Provisional application No. 61/369,492, filed on Jul. 30, 2010.

(51) Int. Cl. C12P 21/00; C12N 15/52

(52) U.S. Cl. CPC C12P 21/005; C12N 15/52
USPC 435/69.1; 435/320.1; 435/254.2

(57) ABSTRACT

A Yeast Artificial Chromosome (YAC) directing the expression of one or more activities of the humanized glycosylation pathway is provided. The said YAC comprises one or more expression cassettes for fusion proteins of heterologous glycosylation pathway and an ER/Golgi retention sequence. The invention also relates to new yeast cells which contain the said YAC. Finally, the invention also provides a method for producing recombinant target glycoproteins.

Yeasts are widely used for the production of recombinant proteins of biological interest because of the established expression system, and it can be easily grown in large quantities. For example, Saccharomyces cerevisiae, Pichia pastoris, Yarrowia lipolytica have all been used for the production of high-molecular weight therapeutics such as growth factors, cytokines, etc. These secretory proteins undergo post-translational modifications including limited proteolysis, folding, disulfide bond formation, phosphorylation and glycosylation. Yeast is thus a preferable host for the production of glycoproteins such as human erythropoietin and alpha-1-antitrypsin.

The first Yeast Artificial Chromosomes (YAC) were described at the beginning of the 1980s (Murray and Szostak, Nature, 305(5931): 189-93, 1983). They were first used to study chromatin organization and chromosome stability (centromere function, segregation during mitosis etc). Since they can accept very long DNA fragments, they have been used to make DNA libraries (Riethman et al, Proc Natl Acad Sci USA, 86(16): 6240-6244, 1989; Chartier et al., Nat Genet., 1(2): 132-136, 1992; Palmieri et al, Gene, 188(2): 169-74, 1997), which were then used in functional studies. For example, YACs were used to clone human telomeres by functional complementation in yeast (Cross et al., Nature, 338(6218): 771-774, 1989; Cheng and Smith, Genet Anal Tech Appl., 7(5): 119-25, 1990) or to determine kinetochore function. These constructions have also proved to be very useful tools for tagging, analyzing (Schlessinger, Trends Genet., 6(8):248: 255-258, 1990) as well as studying the evolution and the organization of complex genomes (Kouprina and Larionov, FEMS Microbiol Rev, 27(5): 629-649, 2003).

The introduction of cassettes conferring resistance to antibiotics such as neomycin has permitted the use of YACs in mammal cells, thus confirming the previous complementation results (Cross et al., Nucl. Acids Res., 18(22): 6649-57, 1990; Srivastava and Schlessinger, Gene, 103(1): 53-59, 1991). YACs have thus been used for expressing proteins of interest in mammal cells, such as ES cells (WO 93/05165). Such YACs can be constructed by using the yeast endogenous recombination and/or repair pathways (WO 95/03400; WO 96/14436).

In addition to these uses, YACs have been used as recipient of several expression cassettes containing heterologous gene sequences which were mixed randomly in order to obtain new metabolites and diverse natural products (WO 2004/016791). For example, this approach has led to a new pathway for flavonoid biosynthesis, thus converting the yeast metabolites phenylalanine and/or tyrosine into flavonoids, normally only produced by plants (Naesby et al., Microb. Cell Fact., 8: 45-56, 2009).

On the other hand, a YAC, because it can accept numerous and/or long DNA fragments, can be used to introduce a whole metabolic pathway in a yeast cell, thus leading to a host cell with new functional properties.

Therapeutic proteins such as erythropoietin or antibodies are glycosylated. Glycosylation is essential both for the protein's function and for their pharmacological properties. For example, the antibody-dependent cellular cytotoxicity (ADCC) of therapeutic antibodies is correlated with an absence of fucosylation of said antibody (see e.g. WO 00/61739, Shields et al., J Biol Chem., 277(30): 26733-26740, 2002, Mori et al., Cytotechnology, 55(2-3): 109-114, 2007, Shinkawa et al., J Biol Chem., 278(5): 3466-73, 2003, WO 03/035835, Chowdury and Wu, Methods, 36(1): 11-24, 2005; Teillaud, Expert Opin Biol Ther., 5(Suppl 1): S15-27, 2005; Presta, Adv Drug Deliv Rev., 58(5-6): 640-656, 2006), while sialylation affects absorption, serum half-life, and clearance from the serum, as well as the physical, chemical and immunogenic properties of the respective glycoprotein (Byrne et. al., Drug Discov Today, 12(7-8): 319-326; Staldmann et al., J Clin Immunol, 30 (Suppl 1): S15-S19, 2010). In addition, the glycosylation of a protein affects its immunogenicity, potentially leading to problems for the patient and thus reducing the protein's therapeutic efficacy (J Immunotoxicol., 3(3): 111-113, 2006).

In order to produce glycoproteins with an optimal N- or O-glycosylation, numerous technical solutions have been proposed. For example, it has been proposed to add glycan structures in vitro by addition of sugar residues such as galactose, glucose, fucose or sialic acid by various glycosyltransferases, or by suppression of specific sugar residues, e.g. elimination of mannose residues by mannosidases (WO 03/031464). However, this method is difficult to use on an industrial scale, since it involves several successive steps for a sequential modification of several oligosaccharides present on the same glycoprotein. At each step, the reaction must be tightly controlled in order to obtain homogenous glycan structures on the recipient protein. Moreover, the use of purified enzymes does not appear to be a viable economic solution. The same problems arise with chemical coupling techniques, like the ones described in WO 2006/106348 and WO 2005/000862. They involve multiple tedious reactions, with protection/deprotection steps and numerous controls. When the same glycoprotein carries several oligosaccharide chains, there is a high risk that sequential reactions lead to undesired, heterogeneous modifications.

Another approach is to use mammalian cell lines such as YB2/0 (WO 01/77181) or a genetically-modified CHO (WO 03/055993) which do not add any fucose residues on the Fc domain of antibodies, thus leading to a 100-fold increase of ADCC activity. However, these technologies are only useful for the production of antibodies.

Recently, it has been proposed to produce in yeast or unicellular filamentous fungi by transforming these microorganisms with plasmids expressing mannosidases and several glycosyltransferases (see e.g. WO 01/4522, WO 02/00879, WO 02/00856). However, up to this day, it has not been demonstrated that these microorganisms are stable throughout time in a high-capacity fermentor. It is therefore unknown whether such cell lines could be reliably used for the production of clinical lots.

Human erythropoietin (HuEPO) is a 166-amino acid glycoprotein which contains 3 N-glycosylation sites at residues Asn-24, Asn-38 and Asn-83 and one mucin O-glycosylation site on position Ser-126. Since oligosaccharide chains make up to 40% of its molecular weight, EPO is a particularly relevant model for studying N-glycosylation. When compared to the urinary form of EPO (uHuEPO), a recombinant EPO (rHuEPO) expressed in CHO cells or in BHK cells displayed different N-glycan structures (Takeuchi et al, J Biol Chem., 263(8): 3657-63, 1988; Sasaki et al., Biochemistry, 27(23): 8618-8626, 1988; Tsuda et al., Biochemistry, 27(15): 5646-5654, 1988; Nimtz et al., Eur J Biochem., 213(1): 39-56, 1993; Rahbek-Nielsen et al., J Mass Spectrom., 32(9): 948-958, 1997). These differences may not have much influence on the protein in vitro, but they lead to dramatic differences in activity in vivo (Higuchi et al, J Biol Chem., 267(11): 7703-7709, 1992).

In order to obtain a protein carrying glycan structures designed for optimal in vivo activity, the present inventors have previously expressed rHuEPO in genetically-modified yeasts (WO 2008/095797). Such strains led to strong expression of proteins with homogenous and well-characterized glycosylation patterns. These yeasts were constructed by insertion of expression cassettes containing various fusions of mammalian glycosylation enzymes with targeting sequences at various locations in the genome. However, constructing new strains can be long and tedious. Moreover, such a construction necessitates the inactivation of numerous auxotrophic markers, which makes the resulting strain less healthy and probably not robust enough as an industrial strain.

Thus there is a need for a yeast cell capable of adding complex N-glycan structures to a target protein and capable of growing robustly in fermentors.

The inventors have now found that it is possible to construct a Yeast Artificial Chromosome (YAC) for the expression of one or more mammalian N-glycosylation enzymes. The construction of the said YAC can be performed quickly and easily, by regular cloning techniques, thus allowing the skilled person to obtain any desired combination of enzymes. The YAC of the invention can then be introduced in any host cell in order to obtain cells capable of adding human-like N-glycan structures. Moreover, the YAC of the invention shows the stability required for robust growth in fermentors.

A yeast according to the present invention is any type of yeast which is capable of being used for large scale production of heterologous proteins. The yeast of the invention thus comprises such species as Saccharomyces cerevisiae, Saccharomyces sp., Hansenula polymorpha, Schizzosaccharomyces pombe, Yarrowia lipolytica, Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Kluyveromyces sp., Kluyveromyces lactis, Candida albicans. Preferably, the yeast of the invention is Saccharomyces cerevisiae. The expression “yeast cell”, “yeast strain”, “yeast culture” are used interchangeably and all such designations include progeny. Thus the words “transformants” and “transformed cells” include the primary subject cells and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

As used herein, the term “N-glycan” refers to an N-linked oligosaccharide, e.g., one that is attached by an asparagine-N-acetylglucosamine linkage to an asparagine residue of a polypeptide. N-glycans have a common pentasaccharide core of Man₃GlcNAc₂ (“Man” refers to mannose; “Glc” refers to glucose; and “NAc” refers to N-acetyl; GlcNAc refers to N-acetylglucosamine). The term “trimannose core” used with respect to the N-glycan also refers to the structure Man₃GlcNAc₂ (“Man₃”). The term “pentamannose core” or “Mannose-₅ core” or “Man₅” used with respect to the N-glycan refers to the structure Man₅GlcNAc₂.

N-glycans differ with respect to the number and the nature of branches (antennae) comprising peripheral sugars (e.g., GlcNAc, galactose, fucose, and sialic acid) that are attached to the Man₃ core structure. N-glycans are classified according to their branched constituents (e.g., high mannose, complex or hybrid). A “high mannose” type N-glycan comprises at least 5 mannose residues. A “complex” type N-glycan typically has at least one GlcNAc attached to the 1,3 mannose arm and at least one GlcNAc attached to the 1,6 mannose arm of the trimannose core. Complex N-glycans may also have galactose (“Gal”) residues that are optionally modified with sialic acid or derivatives (“NeuAc”, where “Neu” refers to neuraminic acid and “Ac” refers to acetyl). A complex N-glycan typically has at least one branch that terminates in an oligosaccharide such as, for example: NeuNAc-; NeuAcα2-6GalNAcα1-; NeuAcα2-3Ga1β1-3GalNAcα1-; NeuAcα2-3/6Galβ1-4GlcNAcβ1-; GlcNAcα1-4Galβ1-(mucins only); Fucα1-2Galβ1-(blood group H). Sulfate esters can occur on galactose, GalNAc, and GlcNAc residues, and phosphate esters can occur on mannose residues. NeuAc (Neu: neuraminic acid; Ac: acetyl) can be O-acetylated or replaced by NeuGl (N-glycolylneuraminic acid). Complex N-glycans may also have intrachain substitutions comprising “bisecting” GlcNAc and core fucose (“Fuc”). A “hybrid” N-glycan has at least one GlcNAc on the terminal of the 1,3 mannose arm of the trimannose core and zero or more mannoses on the 1,6 mannose arm of the trimannose core.

The central part of the repertoire of human glycosylation reactions requires the sequential removal of mannose by two distinct mannosidases (i.e., α-1,2-mannosidase and mannosidase II), the addition of N-acetylglucosamine (by N-acetylglucosaminyl transferase I and II), the addition of galactose (by β-1,4-galactosyltransferase), and finally the addition of sialic acid by sialyltransferases. Other reactions may be controlled by additional enzymes, such as e.g. N-acetylglucosaminyl transferase III, IV, and V, or fucosyl transferase, in order to produce the various combinations of complex N-glycan types. To reconstitute the mammalian glycosylation pathway in yeast, all these enzymes need to be expressed and localized to the ER and/or the Golgi so that they can act sequentially and produce a fully glycosylated glycoprotein.

Eukaryotic protein N-glycosylation occurs in the endoplasmic reticulum (ER) lumen and Golgi apparatus. The process begins with a flip of a branched dolichol-linked oligosaccharide, Man₅GlcNAc₂, synthesized in the cytoplasm, into the ER lumen to form a core oligosaccharide, Glc₃Man₉GlcNAc₂. The oligosaccharide is then transferred to an asparagine residue of the N-glycosylation consensus sequence on the nascent polypeptide chain, and sequentially trimmed by α-glucosidases I and II, which remove the terminal glucose residues, and α-mannosidase, which cleaves a terminal mannose residue. The resultant oligosaccharide, Man₈GlcNAc₂, is the junction intermediate that may either be further trimmed to yield Man₅GlcNAc₂, an original substrate leading to a complex-type structure in higher eukaryotes including mammalian cells, or extended by the addition of a mannose residue to yield Man₉GlcNAc₂ in lower eukaryote, in the Golgi apparatus.

In a first aspect of the invention, a YAC (Yeast Artificial Chromosome) is provided which carries all the genes encoding the enzymes of a whole metabolic pathway. This YAC can be used to reconstitute the said metabolic pathway in yeast.

In a preferred embodiment, the said metabolic pathway is the mammalian glycosylation pathway.

According to this embodiment, the YAC of the invention carries expression cassettes for the expression of one or more mammalian glycosylation enzymes. As used herein, a “YAC” or “Yeast Artificial Chromosome” (the two terms are synonymous and should be construed similarly for the purpose of the present invention) refers to a vector containing all the structural elements of a yeast chromosome. The term “vector” as used herein is intended to refer to a nucleic acid molecule capable of transporting another nucleic acid to which it has been linked.

A YAC as used herein thus refers to a vector, preferably linear, which contains one yeast replication origin, a centromere, and two telomeric sequences. It is also preferable to provide each construct with at least one selectable marker, such as a gene to impart drug resistance or to complement a host metabolic lesion. The presence of the marker is useful in the subsequent selection of transformants; for example, in yeast the URA3, HIS3, LYS2, TRP1, SUC2, G418, BLA, HPH, or SH BLE genes may be used. A multitude of selectable markers are known and available for use in yeast, fungi, plant, insect, mammalian and other eukaryotic host cells.

The YAC of the invention also comprises one or more cassettes for expression of heterologous glycosylation enzymes in yeast. The said enzymes thus include one or more activities of α-mannosidase (α-mannosidase I or α-1,2-mannosidase; α-mannosidase II), N-acetylglucosaminyl transferase (GnT-I, GnT-II, GnT-III, GnT-IV, GnT-V)I, galactosyl transferase I (GalT); fucosyl transferase (FucT), sialyltransferase (SiaT), UDP-N-acetylglucosamine-2-epimerase/N-acetylmannosamine kinase (GNE), N-acetylneuraminate-9-phosphate synthase (SPS), cytidine monophosphate N-acetylneuraminic acid synthase (CSS), sialic acid synthase, CMP-sialic acid synthase, and the like. Such enzymes have been extensively characterized over the years. The genes encoding said enzymes have also been cloned and studied. One could cite for example the gene encoding a Caenorhabditis elegans α-1,2-mannosidase (ZC410.3, an(9)-alpha-mannosidase, Accession number: NM_{—}069176); the gene encoding a murine mannosidase II (Man2a1, Accession number: NM_{—}008549.1); the gene encoding a human N-acetylglucosaminyl transferase I (MGAT1, Accession number: NM_{—}001114620.1); the gene encoding a human N-acetylglucosaminyl transferase II (MGAT2, Accession number: NM_{—}002408.3); the gene encoding a murine N-acetylglucosaminyl transferase III (MGAT3, Accession number: NM_{—}010795.3); the gene encoding the human galactosyl transferase I (B4GALT1, Accession number: NM_{—}001497.3); the gene encoding the human sialyl transferase (ST3GAL4, Accession number: NM_{—}006278); the gene encoding a human UDP-N-acetylglucosamine-2-epimerase/N-acetylmannosamine kinase (GNE, Accession number: NM_{—}001128227); the gene encoding a human N-acetylneuraminate-9-phosphate synthase (NANS, Accession number: NM_{—}018946.3); the gene encoding a human cytidine monophosphate N-acetylneuraminic acid synthase (CMAS, Accession number: NM_{—}018686); the gene encoding a human α-1,6 fucosyltransferase (FUT8, Accession number: NM_{—}178156), the gene encoding a bacterial (N. meningitidis), sialic acid synthase (SiaC, Accession number: M95053.1), the gene encoding a bacterial (N. meningitidis) CMP-sialic acid synthase (SiaB, Accession number M95053.1).

Related genes from other species can easily be identified by any of the methods known to the skilled person, e.g. by performing sequence comparisons.

Sequences comparison between two amino acids sequences are usually realized by comparing these sequences that have been previously aligned according to the best alignment; this comparison is realized on segments of comparison in order to identify and compare the local regions of similarity. The best sequences alignment to perform comparison can be realized, beside by a manual way, by using the global homology algorithm developed by Smith and Waterman (Ad. App. Math., 2: 482-489, 1981), by using the local homology algorithm developed by Neddleman and Wunsch (J. Mol. Biol., 48: 443-453, 1970), by using the method of similarities developed by Pearson and Lipman (Proc. Natl. Acad. Sci. USA, 85: 2444-2448, 1988), by using computer software using such algorithms (GAP, BESTFIT, BLASTP, BLASTN, FASTA, TFASTA in the Wisconsin Genetics software Package, Genetics Computer Group, 575 Science Dr., Madison, Wis. USA), by using the MUSCLE multiple alignment algorithms (Edgar, Nucl. Acids Res., 32: 1792-1797, 2004). To get the best local alignment, one can preferably used BLAST software, with the BLOSUM 62 matrix, or the PAM 30 matrix. The identity percentage between two sequences of amino acids is determined by comparing these two sequences optimally aligned, the amino acids sequences being able to comprise additions or deletions in respect to the reference sequence in order to get the optimal alignment between these two sequences. The percentage of identity is calculated by determining the number of identical position between these two sequences, and dividing this number by the total number of compared positions, and by multiplying the result obtained by 100 to get the percentage of identity between these two sequences.

In addition, a number of publications have also described related enzymes in other species, from which the skilled person can derive the sequence of a gene of interest (see e.g. WO 01/25406; Kumar et al., Proc. Natl. Acad. Sci. U.S.A., 87: 9948-9952, 1990; Sarkar et al., Proc. Natl. Acad. Sci. U.S.A, 88: 234-238, 1991; D'Agostero et al., Eur. J. Biochem., 183: 211-217, 1989; Masri et al., Biochem. Biophys. Res. Commun., 157: 657, 1988; Wang et al., Glycobiology, 1:25-31, 1990; Lal et al., J. Biol. Chem., 269: 9872-9881, 1984; Herscovics et al., J. Biol. Chem., 269: 9864-9871, 1984; Kumar et al., Glycobiology, 2: 383-393, 1992; Nishikawa et al., J. Biol. Chem., 263: 8270-8281, 1988; Barker et al., J. Biol. Chem., 247: 7135, 1972; Yoon et al., Glycobiology, 2: 161-168, 1992; Masibay et al., Proc. Natl. Acad. Sci., 86: 5733-5737, 1989; Aoki et al., EMBO J., 9: 3171, 1990; Krezdorn et al., Eur. J. Biochem., 212: 113-120, 1993).

The skilled person would thus be able to easily identify genes encoding each of the activities involved in mammalian glycosylation.

The person of skills in the art will also realize that, depending on the source of the gene and of the cell used for expression, a codon optimization may be helpful to increase the expression of the encoded bi-functional protein. By “codon optimization”, it is referred to the alterations to the coding sequences for the bacterial enzyme which improve the sequences for codon usage in the yeast host cell. Many bacteria, plants, or mammals use a large number of codons which are not so frequently used in yeast. By changing these to correspond to commonly used yeast codons, increased expression of the bi-functional enzyme in the yeast cell of the invention can be achieved. Codon usage tables are known in the art for yeast cells, as well as for a variety of other organisms.

It is already well known that the mammalian N-glycosylation enzymes work in a sequential manner, as the glycoprotein proceeds from synthesis in the ER to full maturation in the late Golgi. In order to reconstitute the mammalian expression system in yeast, it is necessary to target the mammalian N-glycosylation activities to the Golgi or the ER, as required. This can be achieved by replacing the targeting sequence of each of these proteins with a sequence capable of targeting the desired enzyme to the correct cellular compartment. Of course, it will easily be understood that, if the targeting enzyme of a specific enzyme is functional in yeast and is capable of addressing the said enzyme to the Golgi and/or the ER, there is no need to replace this sequence. Targeting sequences are well known and described in the scientific literature and public databases. The targeting sequence (or retention sequence; as used herein these two terms have the same meaning and should be construed similarly) according to the present invention is a peptide sequence which directs a protein having such sequence to be transported to and retained in a specific cellular compartment. Preferably, the said cellular compartment is the Golgi or the ER. Multiple choices of ER or Golgi targeting signals are available to the skilled person, e.g. the HDEL endoplasmic reticulum retention/retrieval sequence or the targeting signals of the Och1, Mns1, Mnn1, Ktr1, Kre2, Mnn9 or Mnn2 proteins of Saccharomyces cerevisiae. The sequences for these genes, as well as the sequence of any yeast gene can be found at the Saccharomyces genome database web site (http://www.yeastgenome.org/).

It is therefore an object of the invention to provide a YAC comprising one or more expression cassette, said expression cassette encoding a fusion of a heterologous glycosylation enzyme and of an ER/Golgi retention sequence.

According to the invention, the said fusion has been carefully designed before being constructed. The fusions of the invention thus contrast to the prior art which teaches the screening of libraries of random fusions in order to find the one which correctly localizes a glycosylation activity to the correct cellular compartment.

The term “fusion protein” refers to a polypeptide comprising a polypeptide or fragment coupled to heterologous amino acid sequences. Fusion proteins are useful because they can be constructed to contain two or more desired functional elements from two or more different proteins. A fusion protein comprises at least 10 contiguous amino acids from a polypeptide of interest, more preferably at least 20 or 30 amino acids, even more preferably at least 40, 50 or 60 amino acids, yet more preferably at least 75, 100 or 125 amino acids. Fusion proteins can be produced recombinantly by constructing a nucleic acid sequence which encodes the polypeptide or a fragment thereof in-frame with a nucleic acid sequence encoding a different protein or peptide and then expressing the fusion protein. Alternatively, a fusion protein can be produced chemically by crosslinking the polypeptide or a fragment thereof to another protein.

In addition, the said YAC of the invention may advantageously contain transporters for various activated oligosaccharide precursors such as UDP-galactose, CMP-N-acetylneuraminic acid, UDP-GlcNAc, or GDP-Fucose. Said transporters include the CMP-sialic acid transporter (CST), and the like, and the group of sugar nucleotide transporters such as the UDP-GlcNAc transporter, UDP-Gal transporter, GDP-Fucose transporter and CMP-sialic acid transporter. The genes encoding these transporters have been cloned and sequenced in a number of species. For example, one could cite the gene encoding a human UDP-GlcNAc transporter (SLC35A3, Accession number: NM_{—}012243); the gene encoding the fission yeast UDP-Galactose transporter (Gms1, Accession number: NM_{—}001023033.1); the gene encoding a murine CMP-sialic acid transporter (Slc35A1, Accession number: NM_{—}011895.3); the gene encoding a human CMP-sialic acid transporter (SLC35A1; Accession number: NM_{—}006416); and the gene encoding a human GDP-fucose transporter (SLC35C1; Accession number: NM_{—}018389). Thus, in a preferred embodiment, the said YAC of the invention may comprise one or more expression cassettes for transporters, said transporters being selected in the group consisting of CMP-sialic acid transporter, UDP-GlcNAc transporter, UDP-Gal transporter and GDP-Fucose transporter.

Expression cassettes according the invention contain all the necessary sequences for directing expression of the said fusion protein. These regulatory elements may comprise a promoter, a ribosome initiation site, an initiation codon, a stop codon, a polyadenylation signal and a terminator. In addition, enhancers are often required for gene expression. It is necessary that these elements be operable linked to the sequence that encodes the desired proteins. “Operatively linked” expression control sequences refers to a linkage in which the expression control sequence is contiguous with the gene of interest to control the gene of interest, as well as expression control sequences that act in trans or at a distance to control the gene of interest.

Initiation and stop codons are generally considered to be part of a nucleotide sequence that encodes the desired protein. However, it is necessary that these elements are functional in the cell in which the gene construct is introduced. The initiation and termination codons must be in frame with the coding sequence.

Promoters necessary for expressing a gene include constitutive expression promoters such as GAPDH, PGK and the like and inducible expression promoters such as GAL1, CUP1 and the like without any particular limitation. The said promoters can be endogenous promoters, i.e. promoters from the same yeast species in which the heterologous N-glycosylation enzymes are expressed. Alternatively, they can be from another species, the only requirement is that the said promoters are functional in yeast. As an example, the promoter necessary for expressing one of the genes may be chosen in the group comprising of pGAPDH, pGAL1, pGAL10, pPGK, pMET25, pADH1, pPMA1, pADH2, pPYK1, pPGK, pENO, pPHO5, pCUP1, pPET56, pTEF2, pTCM1 the said group also comprising the heterologous promoters pTEF pnmt1, padh2 (both from Schizzosaccharomyces pombe), pSV40, pCaMV, pGRE, pARE, pICL (Candida tropicalis). Terminators are selected in the group comprising CYC1, TEF, PGK, PHO5, URA3, ADH1, PDI1, KAR2, TPI1, TRP1, Bip, CaMV35S, ICL and ADH2.

These regulatory sequences are widely used in the art. The skilled person will have no difficulty identifying them in databases. For example, the skilled person will consult the Saccharomyces genome database web site (http://www.yeastgenome.org/) for retrieving the budding yeast promoters' and/or terminators' sequences.

In addition, the YAC of the invention may comprise one or more expression cassettes for yeast chaperone proteins. Preferably, these proteins are under the same regulatory sequences as the recombinant heterologous protein which is to be produced in the yeast cell. The expression of these chaperone proteins ensures the correct folding of the expressed heterologous protein.

In a preferred embodiment, the expression cassettes of the invention contain the following:
Cassette 1 contains a gene encoding a fusion of an α-mannosidase I and the HDEL endoplasmic reticulum retention/retrieval sequence under the control of the TDH3 promoter and of the CYC1 terminator.
Cassette 2/3 contains a gene encoding a fusion of a N-acetylglucosaminyl transferase I and the S. cerevisiae Mnn9 retention sequence under the control of the ADH1 promoter and of the TEF terminator, and a UDP-GlcNAc transporter gene under the control of the PGK promoter and of the PGK terminator.
Cassette 4 contains an α-mannosidase II gene under the control of the TEF promoter and of the URA terminator.
Cassette 5 contains a gene encoding a fusion of a N-acetylglucosaminyl transferase II and the S. cerevisiae Mnn9 retention sequence under the control of the PMA1 promoter and the ADH1 terminator.
Cassette 6 contains a gene encoding a fusion of a β-1,4-galactosyltransferase and the S. cerevisiae Mnt1 retention sequence under the control of the CaMV promoter and the PHO5 terminator.
Cassette 7 contains the S. cerevisiae PDI1 and KAR2 genes in divergent orientation with their endogenous terminators, both under the control of the pGAL1/10 promoter.
Cassette 8 contains all the ORFs necessary for the sialylation: SiaC(NeuB) under the control of the PET56 promoter and the TPI1 terminator, SiaB(NeuC) under the control of the SV40 promoter and the URA3 terminator, SLC35A1 under the control of the TEF2 promoter and the CaMV terminator and finally ST3GAL4 under the control of the TCM1 promoter and the ADH2 terminator.

According to a further preferred embodiment, an expression cassette of the invention contains a polynucleotide sequence selected from SEQ ID NOS: 1, 2, 3, 4, 5, 6, and 21.

The YAC of the invention may contain one or more of the above expression cassettes. As will be detailed below, it is very easy to combine different expression cassettes, and thus different glycosylation enzymes, leading to the production of glycoproteins with specific glycosylation patterns. The use of the YAC of the invention is thus much easier and much quicker than the construction of new host cells by insertion of an expression cassette directly into the genome of the cell.

The YAC of the invention can be constructed by inserting one or more expression cassettes into an empty YAC vector. In a preferred embodiment, the said empty YAC vector is a circular DNA molecule. In a further preferred embodiment, the empty YAC vector of the invention comprises the following elements:
One yeast replication origin and one centromere ORI ARS1/CEN4;
2 telomeric sequences TEL;
2 selection markers on each arm: HIS3, TRP1, LYS2, BLA or HPH;
1 selection marker for negative selection of recombinants: URA3;
1 multiple cloning site (upstream of LYS2);
1 E. coli replication origin and 1 ampicillin resistance gene;
4 linearization sites: 2 SacI sites and 2 SfiI sites.

In a further preferred embodiment, the empty YAC vectors were designated pGLY-yac_MCS and pGLY-yac-hph_MCS, and have respectively the sequences of SEQ ID NO: 7 and 20. The empty YAC vectors are represented on FIGS. 1 and 2.

The YAC of the invention is constructed by digesting the empty YAC vector and inserting one or more expression cassettes in the said YAC by any method known to the skilled person. For example, according to one embodiment, the empty YAC vector is digested with a unique restriction enzyme. Alternatively, the said empty YAC vector is digested with at least two restriction enzymes. The expression cassette to be inserted in the YAC contains restriction sites for at least one of the said enzymes at each extremity and is digested. After digestion of the cassette with the said same or compatible enzyme(s), the cassette is ligated into the YAC, then transformed into E. coli. The YAC vectors having received the cassettes are identified by restriction digestion or any other suitable way (e.g. PCR). In a related embodiment, the ligation mixture is directly transformed into yeast. In another embodiment, the YAC vector and the digested cassettes are transformed into yeast (without any prior ligation step). According to this embodiment, the cassettes are inserted into the digested YAC vector by recombination within the yeast cells. Other techniques using the yeast recombination pathway are known to the skilled person (e.g. Larionov et al., Proc. Natl. Acad. Sci. U.S.A., 93: 491-496; WO 95/03400; WO 96/14436).

YACs are preferably linear molecules. In a preferred embodiment, a selection marker is excised by the digestion of the empty YAC vector, thus allowing the counter-selection of the circular YAC vectors.

The YAC of the invention can then be introduced into yeast cells as required. The skilled person will resort to the usual techniques of yeast transformation (e.g. lithium acetate method, electroporation, etc, as described in e.g. Johnston, J. R. (Ed.): Molecular Genetics of Yeast, a Practical Approach. IRL Press, Oxford, 1994; Guthrie, C. and Fink, G. R. (Eds.). Methods in Enzymology, Vol. 194, Guide to Yeast Genetics and Molecular Biology. Acad. Press, NY, 1991; Broach, J. R., Jones, E. W. and Pringle, J. R. (Eds.): The Molecular and Cellular Biology of the Yeast Saccharomyces, Vol. 1. Genome Dynamics, Protein Synthesis, and Energetics. Cold Spring Harbor Laboratory Press, Cold Spring Harbor, N.Y., 1991; Jones, E. W., Pringle, J. R. and Broach, J. R. (Eds.): The Molecular and Cellular Biology of the Yeast Saccharomyces, Vol. 2. Gene Expression. Cold Spring Harbor Laboratory Press, Cold Spring Harbor, N.Y., 1992; Pringle, J. R., Broach, J. R. and Jones, E. W. (Eds.): The Molecular and Cellular Biology of the Yeast Saccharomyces, Vol. 3. Cell cycle and Cell Biology. Cold Spring Harbor Laboratory Press, Cold Spring Harbor, N.Y., 1997) for introducing the said YAC into the recipient yeast.

In particular, the YAC of the invention can be introduced into a yeast cell suitable for glycoprotein expression on an industrial scale.

Accordingly, it is another object of this invention to provide a yeast cell for producing target proteins with appropriate complex glycoforms which is capable of growing robustly in fermentors. The yeast cells of the invention are capable of producing large amounts of target glycoproteins with human-like glycan structures. Moreover, the yeast cell of the invention is stable when grown in large-scale conditions. In addition, should additional mutations arise, the yeast cell of the invention can be easily restored in its original form, as required for the production of clinical form. The present invention relates to genetically modified yeasts for the production of glycoproteins having optimized and homogenous humanized oligosaccharide structures.

A yeast according to the present invention is any type of yeast which is capable of being used for large scale production of heterologous proteins. The yeast of the invention thus comprises such species as Saccharomyces cerevisiae, Saccharomyces sp., Hansenula polymorpha, Schizzosaccharomyces pombe, Yarrowia lipolytica, Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Kluyveromyces sp., Kluyveromyces lactis, Candida albicans. Preferably, the yeast of the invention is Saccharomyces cerevisae.

Whereas human N-glycosylation is of the complex type, built on a tri-mannose core extended with GlcNAc, galactose, and sialic acid, yeast N-glycosylation is of the high mannose type, containing up to 100 or more mannose residues (hypermannosylation). Up to the formation of a Man₈ intermediate in the endoplasmic reticulum (ER), both pathways are identical. However, the pathways diverge after the formation of this intermediate, with yeast enzymes adding more mannose residues whereas the mammalian pathway relies on an alpha-1,2-mannosidase to trim further the mannose residues. In order to obtain complex glycosylation in yeast, it is therefore first necessary to inactivate the endogenous mannosyltransferase activities. Yeasts containing mutations inactivating one or more mannosyltransferases are unable to add mannose residues to the Asn-linked inner oligosaccharide Man₈GlcNAc₂.

In a first embodiment, the invention relates to a yeast cell wherein at least one mannosyltransferase activity is deficient and which contains a YAC as described above. By “mannosyltransferase” it is herein referred to an enzymatic activity which adds mannose residues on a glycoprotein. These activities are well known to the skilled person, the glycosylation pathway in yeasts such as Saccharomyces cerevisiae having been extensively studied (Herscovics and Orlean, FASEB J., 7(6): 540-550, 1993; Munro, FEBS Lett., 498(2-3): 223-227, 2001. Karhinen and Makarow, J. Cell Sci., 117(2): 351-358, 2004). In a preferred embodiment, the mannosyltransferase is selected from the group consisting of the products of the S. cerevisiae genes OCH1, MNN1, MNN4, MNN6, MNN9, TTP1, YGL257c, YNR059w, YIL014w, YJL86w, KRE2, YUR1, KTR1, KTR2, KTR3, KTR4, KTR5, KTR6 and KTR7, or homologs thereof. In a further preferred embodiment, the mannosyltransferase is selected from the group consisting of the products of the S. cerevisiae genes OCH1, MNN1 and MNN9, or homologs thereof. In a yet further preferred embodiment, the mannosyltransferase is the product of the S. cerevisiae OCH1 or a homolog thereof. In another further preferred embodiment, the mannosyltransferase is the product of the S. cerevisiae MNN1 or a homolog thereof. In yet another further preferred embodiment, the mannosyltransferase is the product of the S. cerevisiae MNN9 or a homolog thereof. In an even more preferred embodiment, the yeast of the invention is deficient for the mannosyltransferase encoded by the OCH1 gene and/or for the mannosyltransferase encoded by the MNN1 gene and/or the mannosyltransferase encoded by the MNN9 gene.

A mannosyltransferase activity is deficient in a yeast cell, according to the invention, when the mannosyltransferase activity is substantially absent from the cell. It can result from an interference with the transcription or the translation of the gene encoding the said mannosyltransferase. More preferably, a mannosyltransferase is deficient because of a mutation in the gene encoding the said enzyme. Even more preferably, the mannosyltransferase gene is replaced, partially or totally, by a marker gene. The creation of gene knock-outs is a well-established technique in the yeast and fungal molecular biology community, and can be earned out by anyone of ordinary skill in the art (R Rothstein, Methods in Enzymology, 194: 281-301, 1991). According to a further preferred embodiment of the invention, the marker gene encodes a protein conferring resistance to an antibiotic. Even more preferably, the OCH1 gene is disrupted by a kanamycin resistance cassette and/or the MNN1 gene is disrupted by a hygromycin resistance cassette and/or the MNN9 is disrupted by a phelomycin or a blasticidin or a nourseothricin resistance cassette. An “antibiotic resistance cassette”, as used herein, refers to a polynucleotide comprising a gene which codes for a protein, said protein being capable of conferring resistance to the said antibiotic, i.e. being capable of allowing the host yeast cell to grow in the presence of the antibiotic. The said polynucleotide comprises not only the open reading frame encoding the said protein, but also all the regulatory signals required for its expression, including a promoter, a ribosome initiation site, an initiation codon, a stop codon, a polyadenylation signal and a terminator.

The yeast cell of the invention can be used to add complex N-glycan structures to a heterologous protein expressed in the said yeast.

It is thus also an aspect of the invention to provide a method for producing a recombinant target glycoprotein. According to a particular embodiment, the method of the invention comprises the steps of:

(a) introducing a nucleic acid encoding the recombinant glycoprotein into one of the host cell described above;

(b) expressing the nucleic acid in the host cell to produce the glycoprotein; and

(c) isolating the recombinant glycoprotein from the host cell.

The said glycoprotein can be any protein of interest, in particular a protein of therapeutic interest. Such therapeutic proteins include, without limitation, proteins such as cytokines, interleukines, growth hormones, enzymes, monoclonal antibodies, vaccinal proteins, soluble receptors, and all sorts of other recombinant proteins.

The practice of the invention employs, unless other otherwise indicated, conventional techniques or protein chemistry, molecular virology, microbiology, recombinant DNA technology, and pharmacology, which are within the skill of the art. Such techniques are explained fully in the literature. (See Ausubel et al., Current Protocols in Molecular Biology, Eds., John Wiley & Sons, Inc. New York, 1995; Remington's Pharmaceutical Sciences, 17th ed., Mack Publishing Co., Easton, Pa., 1985; and Sambrook et al., Molecular cloning: A laboratory manual 2nd edition, Cold Spring Harbor Laboratory Press—Cold Spring Harbor, N.Y., USA, 1989; Introduction to Glycobiology, Maureen E. Taylor, Kurt Drickamer, Oxford Univ. Press (2003); Worthington Enzyme Manual, Worthington Biochemical Corp. Freehold, N.J.; Handbook of Biochemistry: Section A Proteins, Vol I 1976 CRC Press; Handbook of Biochemistry: Section A Proteins, Vol II 1976 CRC Press; Essentials of Glycobiology, Cold Spring Harbor Laboratory Press (1999)). The nomenclatures used in connection with, and the laboratory procedures and techniques of, molecular and cellular biology, protein biochemistry, enzymology and medicinal and pharmaceutical chemistry described herein are those well known and commonly used in the art.

Unless defined otherwise, all technical and scientific terms used herein have the same meaning as is commonly understood by one of the skill in the art to which this invention belongs.

Having generally described this invention, a further understanding of characteristics and advantages of the invention can be obtained by reference to certain specific examples and figures which are provided herein for purposes of illustration only and are not intended to be limiting unless otherwise specified.

### FIGURES LEGENDS

FIG. 1: Map of pGLY-yac_MCS

FIG. 2: Map of pGLY-yac-hph_MCS

FIG. 3: Construction of a YAC of the invention

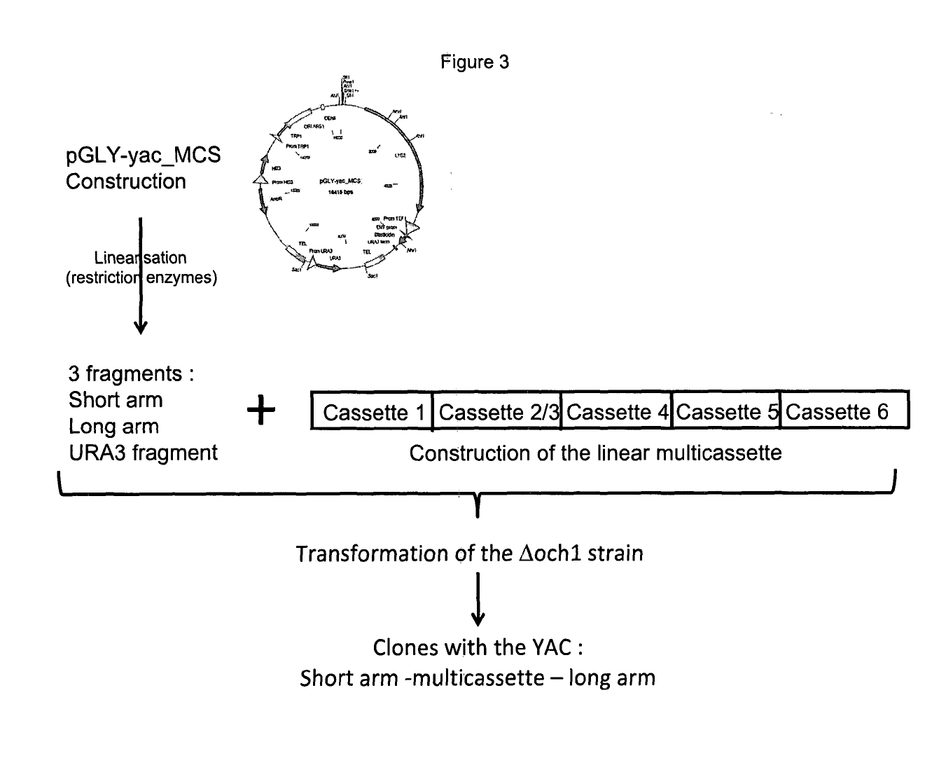

FIG. 4: Validation of Δoch1 strains; A: Analysis of the temperature sensitivity of the Δoch1 transformants; B: PCR analysis of the Δoch1 transformants; C: Expression of rHuEPO in a Δoch1 transformant; D: N-glycan analysis of rHuEPO produced in a Δoch1 transformant.

FIG. 5: RT PCR analysis of expression of integrated ORFs in Gontrand

FIG. 6: Analysis of the YAC stability

FIG. 7: RT PCR analysis of expression of sialylated pathway in Seraphin

### EXAMPLES

Six yeast cells are constructed in order to obtain, on the heterologous protein, the following glycan structures:
GlcNAc₂Man₃GlcNAc₂ (Gontrand strain and DYGorD strain)
Gal₂GlcNAc₂Man₃GlcNAc₂ (George strain and DyoGGène strain)
NeuAc₂Gal₂GlcNAc₂Man₃GlcNAc₂ (Séraphin strain and DrYSSia strain).
In the following examples, the yeast cells are designated by the name of the YAC construct they contain, e;g. the Séraphin cell contains the Séraphin YAC.

### Example 1

### Creation of a och1Δ and/or mnn1Δ and/or mnn9 Δ Host Cell

The kanamycin resistance cassette (containing the KanMX4 cassette, which encodes the enzyme conferring resistance to the said antibiotic) was amplified by PCR and homologous flanking regions to the OCH1 gene were added in both of these ends, specific regions of each strain of S. cerevisiae yeast (see WO 2008/095797). The gene OCH1 is inactivated by inserting this cassette for resistance to an antibiotic, kanamycin. Integration of the gene into the genome of the yeast is accomplished by electroporation and the cassette of interest is then integrated by homologous recombination.

The flanking regions have about forty to one hundred bases and allow integration of the kanamycin resistance cassette within the OCH1 gene in the genome of the yeast.

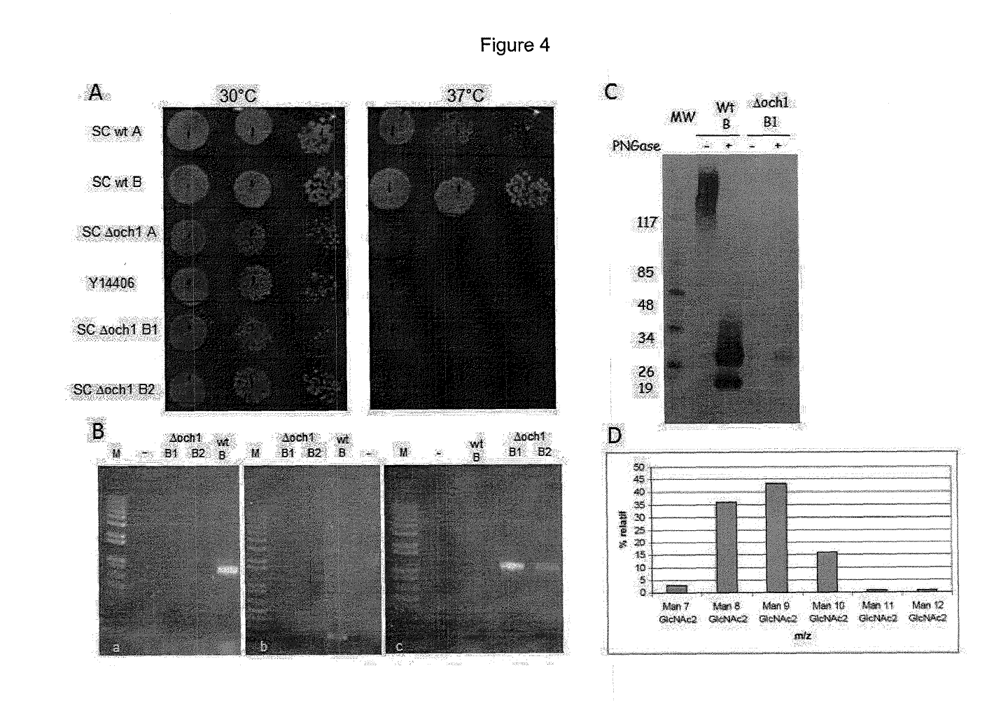

The strains having integrated the gene for resistance to kanamycin are selected on the medium containing 200 μg/mL of kanamycin. A second selection step was performed to use the propriety of growth defect of Δoch1 strains at 37° C. (FIG. 4 A).

We then checked by PCR the integration of the gene for resistance to kanamycin in the OCH1 gene. Genomic DNA of the clones displaying kanamycin resistance was extracted. Oligonucleotides were selected so as to check the presence of kanamycin resistance gene as well as the correct integration of this gene into the OCH1 gene. Primers CR025/BS15 thus led to amplification of a band of the expected size (1237 bp) in the clones having integrated the kanamycin cassette in the OCH1 gene (FIG. 4 Bc). By comparison, no amplification was observed when genomic DNA of wild-type strains was used. On the other hand, PCR reactions using primers hybridizing both within the OCH1 gene led to amplification of DNA fragments for the wild-type, but not for the kanamycin-resistant clones (FIG. 4 Ba BS40/CR004 and Bb CR003/CR004). We conclude that the strains showing kanamycin resistance have integrated the deletion cassette at the correct localization.

The MNN1 gene is replaced by a hygromycin resistance deletion cassette (the said cassette comprises a hph gene, which product is responsible for conferring resistance to the host cells) by following the same method. Likewise, the MNN9 gene is deleted by a blasticidin resistance cassette or a phleomycin resistance cassette or a nourseothricin resistance cassette (comprising the nat1 gene, which product is the nourseothricin acetyltransferase enzyme).

The activity of the Och1 enzyme may be detected by an assay in vitro. Prior studies have shown that the best acceptor for transfer of mannose by the Och1 enzyme is Man₈GlcNAc₂. From microsomal fractions of yeasts (100 μg of proteins) or from a lysate of total proteins (200 μg), the transfer activity of mannose in the alpha-1,6 position on a Man₈GlcNAc₂ structure is measured. For this, the Man₈GlcNAc₂ coupled to an amino-pyridine group (M₈GN₂-AP) is used as an acceptor and the GDP-mannose marked with [¹⁴C]-mannose as a donor molecule of radioactive mannose. The microsomes or the proteins are incubated with the donor (radioactive GDP-mannose), the acceptor (Man₈GlcN₂-AP) and deoxymannojirimycin (inhibitor of mannosidase I) in a buffered medium with controlled pH. After 30 minutes of incubation at 30° C., chloroform and methanol are added to the reaction medium in order to obtain a proportion of CHCl₃/MeOH/H₂O of 3:2:1 (v/v/v). The upper phase corresponding to the aqueous phase, contains Man₈GlcNAc₂-AP, radioactive Man₉GlcNAc₂-AP and GDP-[¹⁴C]-mannose. Once dried, the samples are taken up in 100 μL of H₂O/1% acetic acid and passed over a Sep-Pak C18 (Waters) column, conditioned beforehand in order to separate GDP-mannose from the formed radioactive Man₉GlcNAc₂-AP (the AP group allows this compound to be retained on the C18 columns). By eluting with H₂O/1% acetic acid (20 mL) and then with 20% methanol/1% acetic acid (4 mL), the different fractions may be recovered and counted with the scintillation counter.

### Heterologous Protein Production and Glycan Analysis:

The modified yeast strains are transformed by an expression vector that contains EPO sequence under a galactose-inducible promoter. Yeasts used for producing human EPO are first of all cultivated in a uracil drop out YNB medium, 2% glucose until an OD₆₀₀>12 is reached. After 24-48 hours of culture, 2% galactose is added to the culture in order to induce the production of our protein of interest. Samples are taken after 0, 24 hours of induction.

Yeast cells are eliminated by centrifugation. The supernatant is first buffered at pH 7.4 by adding Imidazole 5 mM, Tris HCl 1 M pH=9, until the desired pH is reached. The supernatant is then filtered on 0.8 μm and 0.45 μm before being loaded on a HisTrap HP 1 mL column (GE Healthcare). EPO is purified according to the manufacturer's instructions (equilibration buffer: Tris HCl 20 mM, NaCl 0.5 M, Imidazole 5 mM, pH=7.4; elution buffer: Tris HCl 20 mM, NaCl 0.5 M, Imidazole 0.5 M, pH=7.4).

The produced EPO is recovered in the eluate. The proteins eluted from the column are analyzed by SDS-PAGE electrophoresis on 12% acrylamide gel.

After migration of the SDS-PAGE gel, analysis of the proteins is accomplished either by staining with Coomassie blue or by western blot. For western blotting, the total proteins are transferred onto a nitrocellulose membrane in order to proceed with detection by the anti-EPO antibody (R&D Systems). After the transfer, the membrane is saturated with a blocking solution (PBS, 5% fat milk) for 1 hour. The membrane is then put into contact with the anti-EPO antibody solution (dilution 1:1000) for 1 hour. After three rinses with 0.05% Tween 20-PBS the membrane is put into contact with the secondary anti-mouse-HRP antibody in order to proceed with colorimetric detection (FIG. 4 C).

A protein at about 35 kDa can thus be detected after deglycosylation. This protein is the major protein detected by Coomassie staining and is revealed by an anti-EPO antibody in a western blot analysis.

N-glycan analysis after PNGase treatment showed that the rHuEPO produced in the Δoch1 strain carried oligomannosyl glycan structures of the type: Man_{8/9}GlcNAc₂. (FIG. 4 D)

### Example 2

### Construction of the GonTRanD/DYGorD George/DYoGGène and Séraphin/DrYSSia Strains

The sequences containing the genes for the different mannosidases and glycosyltransferases are introduced into the YACs as expression cassettes, each gene being under the control of a different constitutive promoter and terminator. The use of different regulatory elements allows for a good stability of the recombinant YACs. The YACs may also contain the genes encoding two yeast protein chaperones (Pdi1 and Kar2). These genes are under the control of the pGAL1/10 promoter in order to coordinate their expression with the expression of the heterologous protein to be expressed.

The George and DYoGGène's YACs contain cassettes 1-7.

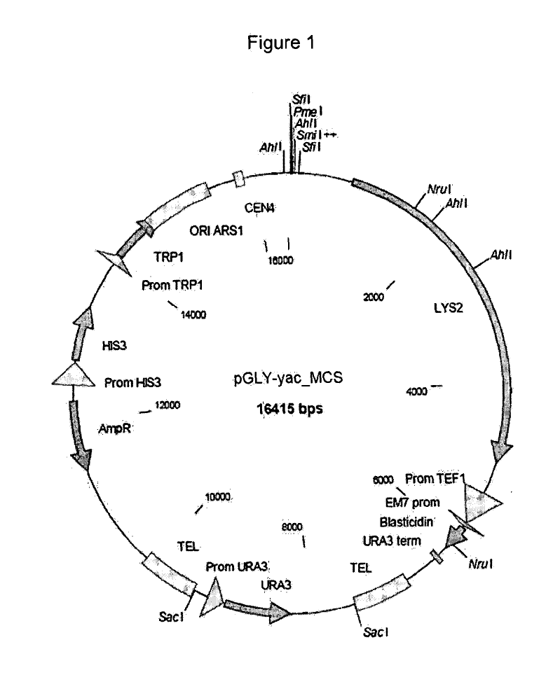

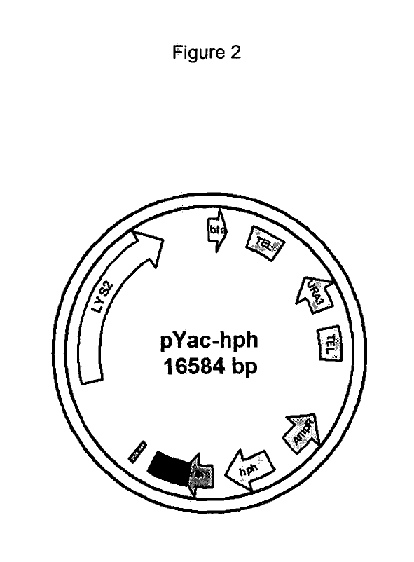

The said YACs are constructed by digesting by SfiI and SacI pGLY-yac_MCS or pGLY-yac-hph_MCS (see FIGS. 1 and 2), respectively. This digestion gives three linear fragments, i.e. the two arms and the URA3 marker.

Each of the 7 cassettes is bordered by SfiI sites. The use of the SfiI restriction site: GGCCNNNN↓NG GCC generates compatible, unique, cohesive ends between the different cassettes and only allows for one type of assembling between the 7 expression cassettes.

Cassette 1:
GGCC ATGC↓A GGCC_GGCC CGTA↓C GGCC
Cassette ⅔:
GGCC CGTA↓C GGCC_GGCC TGAC↓G GGCC
Cassette 4:
GGCC TGAC↓G GGCC_GGCC GCTA↓T GGCC
Cassette 5:
GGCC GCTA↓T GGCC_GGCC ACGC↓T GGCC
Cassette 6:
GGCC ACGC↓T GGCC_GGCC CCTG↓A GGCC
Cassette 7:
GGCC CCTG↓A GGCC_GGCC GACT↓C GGCC
Cassette 8:
GGCC CCTG↓A GGCC_GGCC GACT↓C GGCC

The cassettes are assembled by cloning into an intermediary vector and then the “polycassette” is excised by a new SfiI digestion.

After purification of the corresponding band, the linearized polycassette is transformed in yeast with the linearized pGLY-yac_MCS.

The recipient yeast strain contains the och1::KanMX4 and/or mnn1:hph and/or mnn9::nat1 alleles (see above). Alternatively, the MNN9 gene may be disrupted with the blasticidin or the phleomycin resistance, cassette instead of the nourseothricin resistance cassette.

The said yeast strain is inoculated in 500 mL YPD (1% Yeast Extract, 2% Peptone, 2% D-glucose) at OD₆₀₀=0.1 and is grown until an OD₆₀₀ of between 5.5 and 6.5 is reached.

The cells are centrifuged 5 minutes at 4° C. at 1500 g. The cell pellet is washed twice in cold sterile water (first, with 500 mL, then with 250 mL), before being resuspended in 20 mL of sterile sorbitol 1 M. The cells are centrifuged once more before being resuspended in mL sterile sorbitol 1 M. At this stage, the cells are aliquoted by 80 μL and can be frozen at −80° C. if needed.

Transformation is performed by electroporation. Briefly, the cells are incubated with the DNA (SfiI-SacI digested pGLY-yac_MCS and SfiI digested polycassette) for 5 minutes on ice. A pulse at V=1500 V is given. The cells are immediately resuspended gently in 1 mL cold sterile sorbitol 1 M, and then are incubated for recovery for 1 hour at 30° C. The cells are then plated onto selective medium. In the present case, the selective medium is YNB (0.17% (wt/vol) yeast nitrogen base (without amino acids and ammonium sulfate, YNBww; Difco, Paris, France), 0.5% (wt/vol) NH₄Cl, uracil (0.1 g/L), 0.1% (wt/vol) yeast extract (Bacto-DB), 50 mM phosphate buffer, pH 6.8, and, for solid medium only, 2% agar), containing all the required supplements for the growth of the transformants, except histidine, tryptophan, lysine which are used for positive selection of the transformants+/−blasticidin for selection. On the other hand, the YNB plates contain 5-fluorootic acid (5-FOA) to counter select the circular pGLY-yac_MCS transformants.

The transformants thus growing on these selection plates should all contain a pGLY-yac_MCS YAC wherein the polycassette has been inserted. The presence of the polycassette in the YAC is checked by PCR for each transformant.

The GoNTRanD and DYGoRD's YAC differ from the George and DYoGGène's YACs in that they only contain cassettes 1-5.

The GoNTRanD cells were recovered and the RNA extracted and purified (RNeasy mini kit Qiagen). Each of the RNA samples was divided into two, with one half being treated with an RNase (Sigma-Aldrich) for 30 minutes at room temperature (control for no DNA contamination during the extraction), while the other was left untreated. Reverse transcription was performed on all of the RNA samples, including the RNase-treated negative control. A PCR negative control consisting of water was included in the reactions.

| | | |
|---|---|---|
| 1 μg RNA | | |
| | {close oversize brace} | 5 min 70° C. |
| 0.5 μg oligo dT | | |

60 nmol MgCl₂
10 nmol dNTP
20 U RNase Inhibitor +
buffer RT + reverse transcriptase

The following primers were used in the reverse transcription reactions:

CA027:
(SEQ ID NO. 22)
GGAAAGACGGGTGCAAC
CA028:
(SEQ ID NO. 23)
CCCAACGTCATATAATGATCTGA
CA017:
(SEQ ID NO. 24)
ATGTTCGCCAACCTAAAATACG
CA018:
(SEQ ID NO. 25)
TTACAAGGATGGCTCCAAGG
CA046:
(SEQ ID NO. 26)
TCCAGGGCTACTACAAGA
CR008:
(SEQ ID NO. 27)
CCAGCTCCTTCCGGTCA
CA040:
(SEQ ID NO. 28)
TGGAGAAGATAATTGGAGAT
CA041:
(SEQ ID NO. 29)
GCGGTCTTAGGGAAACATA
CD030:
(SEQ ID NO. 30)
CCCGAATACCTCAGACTG
CD031:
(SEQ ID NO. 31)
ACTCGATCAGCTTCTGATAG

| | K7Y1 | K7Y2-3 | | K7 Y4 | K7Y5 |
|---|---|---|---|---|---|
| | Man I | UDP Glc Nac Tr | GNTI | Man II | GNTII |
| | strain | | | | |
| | CA027-CA028 | CA017-CA018 | CA046-CR008 | CA040-CA041 | CD030-CD031 |
| | 800 pb | 920 pb | 609 pb | 694 pb | 600 pb |
| GoNTranD | 1-2-3-4 | 19-20-21-22 | 37-38-39-40 | 55-56-57-58 | 73-74-75-76 |
| | 5-6-7-8 | 23-24-25-26 | 41-42-43-44 | 59-60-61-62 | 77-78-79-80 |
| | 9-10-11-12 | 27-28-29-30 | 45-46-47-48 | 63-64-65-66 | 81-82-83-84 |
| | 13-14-15-16 | 31-32-33-34 | 49-50-51-52 | 67-68-69-70 | 85-86-87-88 |
| Parental | 17 | 35 | 53 | 71 | 89 |
| control | | | | | |
| Negative | 18 | 36 | 54 | 72 | 90 |
| control | | | | | |

PCR on cDNA was performed in 25 μL containing 12.5 μL of mix Dynazyme, 1.25 μL of each primer (10 pmol/μL), 8 μL H₂O, and 2 μL cDNA. The cDNAs were first denatured for 5′ at 95° C., then subjected to 30 cycles of denaturation of 40″ at 95° C., hybridization for 40″ at 53° C., and elongation for 1′ at 72° C., before elongation was completed for 5′ at 72° C.

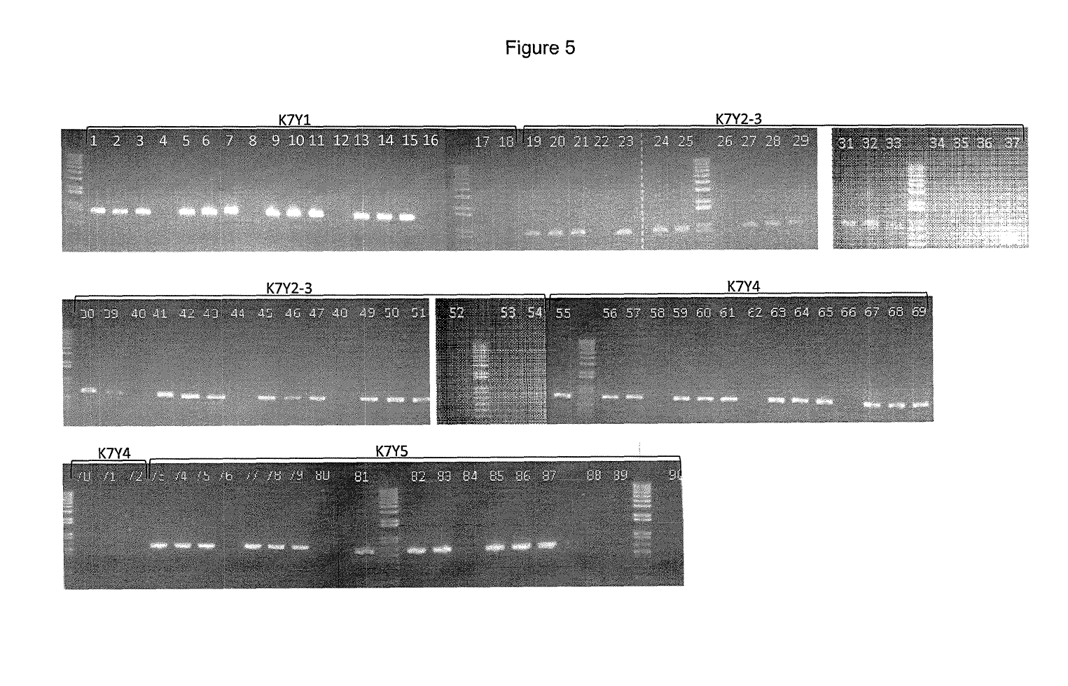

The PCR products were run on an agarose gel to verify the presence of amplification band. The results shown in FIG. 5 demonstrate a specific amplification of bands of the expected size in yeast cultures.

The Séraphin and DrYSSia's YACs differ from George and DYoGGène's YACs in that they also carry the open reading frames for human sialyl transferase ST3GAL4 (NM_{—}006278), murine CMP-sialic acid transporter (NM_{—}011895.3), Neisseria meningitidis CMP-sialic acid synthase (U60146 M95053.1), and N. meningitidis sialic acid synthase (M95053.1). These open reading frames are contained within cassette 8. In addition, these YACs do not contain the cassette 7 (PDI-BIP). The construction of this second series of YACs is performed like the first one.

The Seraphin cells were recovered and the RNA extracted and purified (RNeasy mini kit Qiagen). Each of the RNA samples was divided into two, with one half being treated with an RNase (Sigma-Aldrich) for 30 minutes at room temperature (control for no DNA contamination during the extraction), while the other was left untreated. Reverse transcription was performed on all of the RNA samples, including the RNase-treated negative control. A PCR negative control consisting of water was included in the reactions.

Sialic acid pathway expression

| | cDNA 2 μL | | | |
|---|---|---|---|---|
| | Sia C | SiaB | SLC53A1 | ST3GAL4 |
| | (meningitidis) | (meningitidis) | (mouse) | (human) |
| | CA095- | CB125- | CB144- | CB127- |
| | CA096 | CB126 | CB145 | CB104 |
| | 210 pb | 263 pb | 322 pb | 790 pb |
| Seraphin | 1, 2, 3 | 6, 7, 8 | 11, 12, 13 | 16, 17, 18 |
| Wild type | 4 | 9 | 14 | 19 |
| strain | | | | |
| H2O | 5 | 10 | 15 | 20 |

Negative control (Rnase) in bold

| | | |
|---|---|---|
| 1 μg RNA | | |
| | {close oversize brace} | 5 min 70° C. |
| 0.5 μg oligo dT | | |

60 nmol MgCl₂
10 nmol dNTP
20 U RNase Inhibitor +
buffer RT + reverse transcriptase

The following primers were used in the reverse transcription reactions:

CA095:
(SEQ ID NO. 32)
cagtagctttaggcggttc
CA096:
(SEQ ID NO. 33)
gctacgacagatgcaaagg
CB125:
(SEQ ID NO. 34)
tggcgggttaattgcagaag
CB126:
(SEQ ID NO. 35)
agtggatgatgctccattgg
CB144:
(SEQ ID NO. 36)
aggaactggcgaagttgagt
CB145:
(SEQ ID NO. 37)
actcctgcaaatccagagca
CB127:
(SEQ ID NO. 38)
gcttgaggattatttctggg
CB104:
(SEQ ID NO. 39)
tcagaaggacgtgaggttc

PCR on cDNA was performed in 25 μL containing 12.5 μL of mix Dynazyme, 1.25 μL of each primer (10 pmol/μL), 8 μL H₂O, and 2 μL cDNA. The cDNAs were first denatured for 5′ at 95° C., then subjected to 30 cycles of denaturation of 30″ at 95° C., hybridization for 30″ at 56° C., and elongation for 40″ at 72° C., before elongation was completed for 5′ at 72° C.

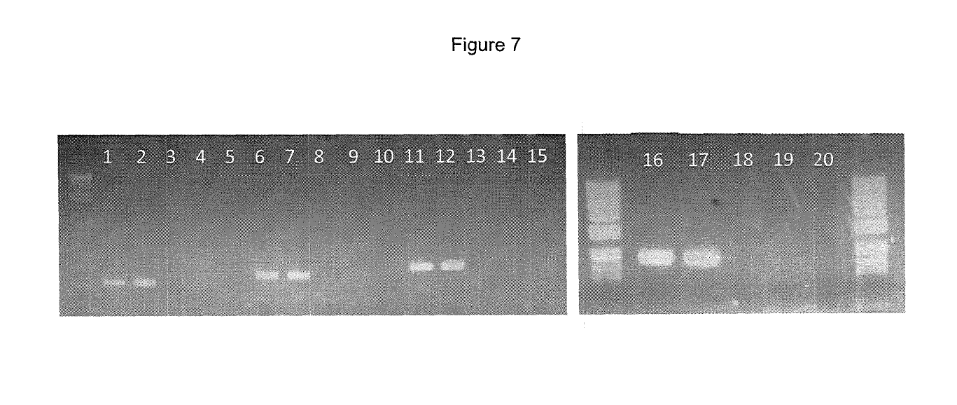

The PCR products were run on an agarose gel to verify the presence of amplification band. The results shown in FIG. 7 demonstrate a specific amplification of bands of the expected size in yeast cultures.

### Example 3

### EPO Expression in the George Strain

The George strain is capable of exclusively producing the N-glycan Gal₂GlcNAc₂Man₃GlcNAc₂, a structure encountered in mammals, described as a glycan of a complex type. The presence of the construction of the relevant YAC and its introduction into a host cells is described above. Each of these steps enters a “package” of verifications consisting of selecting the best producing clone and of maximizing the percentage of chances in order to obtain an exploitable clone.

The plasmid used for the expression of EPO in the modified yeasts contains the promoter Gal1. This promoter is one of the strongest promoters known in S. cerevisiae and is currently used for producing recombinant proteins. This promoter is induced by galactose and repressed by glucose. Indeed, in a culture of S. cerevisiae yeasts in glycerol, addition of galactose allows induction of the GAL genes by about 1,000 times. on the other hand, addition of glucose to the medium represses the activity of the GAL1 promoter. The integrated sequence of human EPO in our plasmid was modified in 5′ by adding a polyhistidine tag in order to facilitate detection and purification of the produced protein.

The yeasts used for producing human EPO are first of all cultivated in a uracil drop out YNB medium, 2% glucose until an OD₆₀₀>12 is reached. After 24-48 hours of culture, 2% galactose is added to the culture in order to induce the production of our protein of interest. Samples are taken after 0, 6, 24 and 48 hours of induction.

Yeast cells are eliminated by centrifugation. The supernatant is first buffered at pH 7.4 by adding Imidazole 5 mM, Tris HCl 1 M pH=9, until the desired pH is reached. The supernatant is then filtered on 0.8 μm and 0.45 μm before being loaded on a HisTrap HP 1 mL column (GE Healthcare). EPO is purified according to the manufacturer's instructions (equilibration buffer: Tris HCl 20 mM, NaCl 0.5 M, Imidazole 5 mM, pH=7.4; elution buffer: Tris HCl 20 mM, NaCl 0.5 M, Imidazole 0.5 M, pH=7.4).

The produced EPO is recovered in the eluate. The proteins eluted from the column are analyzed by SDS-PAGE electrophoresis on 12% acrylamide gel.

After migration of the SDS-PAGE gel, analysis of the proteins is accomplished either by staining with Coomassie blue or by western blot. For western blotting, the total proteins are transferred onto a nitrocellulose membrane in order to proceed with detection by the anti-EPO antibody (R&D Systems). After the transfer, the membrane is saturated with a blocking solution (PBS, 5% fat milk) for 1 hour. The membrane is then put into contact with the anti-EPO antibody solution (dilution 1:1000) for 1 hour. After three rinses with 0.05% Tween 20-PBS the membrane is put into contact with the secondary anti-mouse-HRP antibody in order to proceed with colorimetric detection.

A protein at about 35 kDa can thus be detected. This protein is the major protein detected by Coomassie staining and is revealed by an anti-EPO antibody in a western blot analysis.

Eluted fractions containing EPO are concentrated by centrifugation at 4° C. on Amicon Ultra-15 (Millipore), with a cut-off of 10 kDA. When a volume of about 500 μL is obtained, the amount of purified protein is assayed.

N-glycan analysis after PNGase treatment showed that the rHuEPO produced in the George strain carried complex glycan structures of the type: Gal₂GlcNAc₂Man₃GlcNAc₂.

### Example 4

### YAC Stability

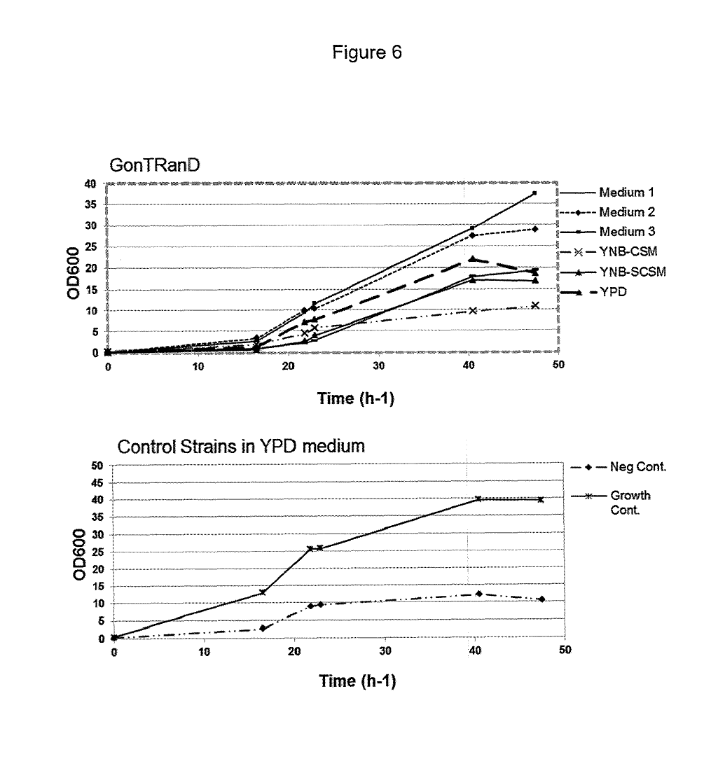

In order to assess the YAC stability, yeast cells carrying the GoNTRanD YAC were grown in selective media or not in a micro-fermentor (BioPod—FIG. 6 A), then plated on several selective agar media (CSM, CSM LYS DO, DO LEU MSC, MSC DO HIS, URA DO CSM, CSM+blasticidin) to get between 40 and 400 colonies. The plates were then incubated 4 days at 30° C. and the colonies counted. Stability tests are performed at 0, 24 and 48 hours of growth in a micro-fermenter.

The FIG. 6B shows the percentage of stability of the YAC in several media (selective or not) in GoNTRanD strain. The percentage of stability is calculated according to the formula: % of stability=((colony number on selective plate)/(colony number on non-selective plate))/100. The negative control is the parental strain of GoNTRanD (same genetic background but without YAC) and the control of growth is a prototrophic strain.

Medium 1: Selective media produced in-house

Medium 2: Non-selective media produced in-house

Medium 3: Non-selective media produced in-house

YNB CSM: Non-selective synthetic medium

YNB S-CSM: Non-selective synthetic medium

YPD: Non-selective complete medium

This artificial chromosome was stable during a production time in non-selective media (FIG. 6 B) and compared to an episomal vector (data not shown). This stability was conserved during scale-up of culture, from micro-fermentor to 5 L-bioreactor. In all the different tests, stability was always slightly increased with our “in-house” growing medium.

Then, the integrity of the YAC was checked by PCR verification of the presence of the 5 ORFs on genomic DNA. All ORFs present on the artificial chromosome could be amplified from a yeast cell grown for 70 hrs in non-selective growth conditions followed by 48 hrs of culture in conditions of production in 5 L-Bioreactor (data not shown).

1. A Yeast Artificial Chromosome (YAC) comprising at least one cassette for expression of heterologous glycosylation enzymes in yeast.

2. The YAC of claim 1, wherein said heterologous glycosylation enzyme is selected from the group consisting of α-mannosidase I (α-1,2-mannosidase), α-mannosidase II, N-acetylglucosaminyl transferase I, N-acetylglucosaminyl transferase II, N-acetylglucosaminyl transferase III, N-acetylglucosaminyl transferase IV, N-acetylglucosaminyl transferase V, galactosyl transferase I, fucosyl transferase, sialyltransferase, UDP-N-acetylglucosamine-2-epimerase/N-acetylmannosamine kinase, N-acetylneuraminate-9-phosphate synthase, cytidine monophosphate N-acetylneuraminic acid synthase, sialic acid synthase, and CMP-sialic acid synthase.

3. The YAC of claim 1, wherein at least one of said expression cassette encodes a fusion protein of the catalytic domain of a heterologous glycosylation enzyme and of an ER/Golgi retention signal.

4. The YAC of claim 1, wherein the retention signal is selected from the group consisting of the HDEL endoplasmic reticulum retention/retrieval sequence and the targeting signals of the Och1, Msn1, Mnn1, Ktr1, Kre2, Mnt 1, Mnn2 and Mnn9 proteins of Saccharomyces cerevisiae.

5. The YAC of claim 1, wherein said YAC further comprises at least one expression cassette for a transporter, said transporter being selected from the group consisting of CMP-sialic acid transporter, UDP-GlcNAc transporter, UDP-Gal transporter and GDP-Fucose transporter.

6. The YAC of claim 1, wherein said YAC further comprises at least one expression cassette for a yeast protein chaperone.

7. The YAC of claim 1, wherein said YAC comprises a promoter selected from the group consisting of pGAPDH, pGAL1, pGAL10, pPGK, pTEF, pMET25, pADH1, pPMA1, pADH2, pPYK1, pPGK, pENO, pPHO5, pCUP1, pPET56, pnmt1, padh2, pSV40, pCaMV, pGRE, pARE pICL, pTEF2 and pTCM1.

8. The YAC of claim 1, wherein said YAC comprises a terminator selected from the group consisting of CYC1, TEF, PGK, PHO5, URA3, ADH1, PDI1, KAR2, TPI1, TRP1, CaMV35S, ADH2 and ICL.

9. The YAC of claim 1, wherein said YAC comprises at least one of:
Cassette 1, said cassette 1 comprising a gene encoding a fusion of an α-mannosidase I and a retention sequence HDEL under control of TDH3 promoter and of CYC1 terminator
Cassette 2/3, said cassette 2/3 comprising a gene encoding a fusion of a N-acetylglucosaminyl transferase I and S. cerevisiae Mnn9 retention sequence under control of ADH1 promoter and of TEF terminator, and a UDP-GlcNAc transporter gene under control of PGK promoter and of PGK terminator
Cassette 4, said cassette 4 comprising an α-mannosidase II gene under control of TEF promoter and of URA terminator
Cassette 5, said cassette 5 comprising a gene encoding a fusion of a N-acetylglucosaminyl transferase II and S. cerevisiae Mnn9 retention sequence under control of PMA1 promoter and ADH1 terminator
Cassette 6, said cassette 6 comprising a gene encoding a fusion of human β-1,4-galactosyltransferase and S. cerevisiae Mnt1 retention sequence under control of CaMV promoter and PHO5 terminator
Cassette 7, said cassette 7 comprising S. cerevisiae PDI1 and KAR2 genes in divergent orientation with endogenous terminators, said terminators being under control of pGAL1/10 promoter
Cassette 8, said cassette 8 comprising SiaC (NeuB) gene under control of PET56 promoter and TPI1 terminator, the SiaB (NeuC) gene under control of SV40 promoter and URA3 terminator, SLC35A1 gene under control of TEF2 promoter and CaMV terminator and the ST3GAL4 gene under control of TCM1 promoter and ADH2 terminator.

10. The YAC of claim 1, wherein said YAC comprises at least one cassette having a sequence selected from the group consisting of SEQ ID N01, SEQ ID NO: 2, SEQ ID NO: 3, SEQ ID NO: 4, SEQ ID NO: 5, SEQ ID NO: 6, and SEQ NO. 21.

11. A method for constructing a YAC according to claim 1, comprising inserting at least one expression cassette into an empty YAC vector.

12. The method of claim 11, wherein said empty YAC vector comprises:
One yeast replication origin and one centromere ORI ARS1/CEN4;
2 telomeric sequences TEL;
2 selection markers on each arm: HIS3, TRP1, LYS2, BLA or HPH
1 selection marker for negative selection of recombinants: URA3;
1 multiple cloning site (upstream of LYS2);
1 E. coli replication origin and 1 ampicillin resistance gene;
4 linearization sites: 2 SacI sites and 2 SfiI sites.

13. The method of claim 11, wherein said empty YAC vector comprises the DNA sequence of SEQ ID NO: 7.

14. A yeast cell for producing a target glycoprotein, wherein said yeast cell comprises a YAC according to claim 1.

15. The yeast cell according to claim 14, wherein said yeast cell is deficient in mannosyltransferase activity.

16. The yeast cell according to claim 14, wherein said yeast cell comprises a deletion of OCH1 gene and/or MNN1 gene and/or MNN9 gene and/or MNN2 gene.

17. The yeast cell according to claim 14, wherein said cell is capable of producing glycoprotein with glycan structure selected from
GlcNAc₂Man₃GlcNAc₂,
Gal₂GlcNAc₂Man₃GlcNAc₂ and
NeuAc₂ Gal₂GlcNAc₂Man₃GlcNAc₂.

18. The yeast cell according to claim 14, wherein said yeast is Saccharomyces cerevisiae.

19. A method for producing a recombinant target glycoprotein, said method comprising:
(a) introducing a nucleic acid encoding the recombinant glycoprotein into said yeast cell of claim 14;
(b) expressing the nucleic acid in the host cell to produce the glycoprotein; and
(c) isolating the recombinant glycoprotein from the host cell.


SEQUENCE LISTING (39 sequences)

SEQ ID NO: 1 - DNA, 2593 bp, Artificial Sequence
  Sequence of the expression cassette 1
tcgagtttatcattatcaatactagccatttcaaagaatacgtaaataattaatagtagtgattttcctaactttatttagtcgaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaatctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagtcttaaaacaccaagaacttagtttcgaataaacacacataaataaacaaaatgatgggcctccgatcacacgaacaacttgtcgtgtgtgtcggagttatgtttcttctgactgtctgcatcacagcgtttttctttcttccgtcaggcggcgctgatctgtatttccgagaagaaaactccgttcacgttagagatgtgcttatcagtttcagagaggaaattcgtcgtaaagagcaaggtgagttacggcggaaagccgaagaagccaatcccattccaattccaaaacctgaaattggagcatcggatgatgcagaaggacgaagaattttcgtgaaacaaatgattaaattcgcatgggacggatatcggaaatatgcctggggggagaatgaattgaggcccaacagtagatcaggacattcttcatcgatatttgggtatggaaagacgggtgcaacaattattgatgctattgatacattgtatttggttggattaaaagaagaatataaagaggccagagactggattgctgattttgatttcaaaacgtctgcgaaaggagatctatcagtttttgaaacaaatatccgattcactggtggcctactctccgcatttgcacttaccggagacaaaatgttcttgaagaaagcagaagatgtggcaactattcttcttccggcttttgaaactccttctggaataccaaattcattaattgatgctcaaacaggaagatccaaaacgtatagttgggcaagcggaaaggcaattctctcggaatacggttcaattcaacttgaattcgattatctctccaatctgactggaaatccagtttttgctcaaaaagctgataaaataagagatgttttaactgcaatggagaaaccagaaggactttatccaatttatattactatggataatccaccaagatggggacaacatcttttctcaatgggtgcaatggctgacagttggtatgaatatctgctcaaacaatggattgccactggtaaaaaagatgatcgcacgaaaagagaatacgaagaagcgatatttgcaatggaaaaacgaatgcttttcaaatcggaacagtcgaatctttggtatttcgcaaaaatgaacggaaatcgcatggaacattcatttgaacatcttgcatgcttttccggtggaatggttgttcttcatgcaatgaatgagaaaaataaaacaatatcagatcattatatgacgttgggaaaagaaattggtcatacatgtcatgaatcgtacgctagatccacaactggaatcggcccagaatccttccaattcacatcgagtgtagaggcaaaaacagaacgtcgtcaggattcatattatattcttcgtcccgaagtcgttgagacatggttctacttgtggagggctacaaaagacgagaaatatcgacaatgggcttgggatcatgttcaaaatttggaggagtattgtaagggcactgccggatactctggaatccgaaacgtctacgaatcgagcccggaacaagatgatgtgcagcagtcattcctcttcgctgagctcttcaaatatctgtatttaattttcagtgaagataacattcttccacttgatcaatgggttttcaataccgaagctcatccattccgcattcggcatcacgacgagttgattgaattggtcgatcaggtattcatgtaattcgttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagct

SEQ ID NO: 2 - DNA, 4167 bp, Artificial Sequence
  Sequence of the expression cassette 2/3
aagaaatgatggtaaatgaaataggaaatcaaggagcatgaaggcaaaagacaaatataagggtcgaacgaaaaataaagtgaaaagtgttgatatgatgtatttggctttgcggcgccgaaaaaacgagtttacgcaattgcacaatcatgctgactctgtggcggacccgcgctcttgccggcccggcgataacgctgggcgtgaggctgtgcccggcggagttttttgcgcctgcattttccaaggtttaccctgcgctaaggggcgagattggagaagcaataagaatgccggttggggttgcgatgatgacgaccacgacaactggtgtcattatttaagttgccgaaagaacctgagtgcatttgcaacatgagtatactagaagaatgagccaagacttgcgagacgcgagtttgccggtggtgcgaacaatagagcgaccatgaccttgaaggtgagacgcgcataaccgctagagtactttgaagaggaaacagcaatagggttgctaccagtataaatagacaggtacatacaacactggaaatggttgtctgtttgagtacgctttcaattcatttgggtgtgcactttattatgttacaatatggaagggaactttacacttctcctatgcacatatattaattaaagtccaatgctagtagagaaggggggtaacacccctccgcgctcttttcatgtcactttctcttgtatcgtaccgcctaagaaagaacccgtgggttaacgcagggcttgtgctgtggggcgctatcctctttgtggcctggaatgccctgctgctcctcttcttctggacgcgcccagcacctggcaggccaccctcagtcagcgctctcgatggcgaccccgccagcctcacccgggaagtgattcgcctggcccaagacgccgaggtggagctggagcggcagcgtgggctgctgcagcagatcggggatgccctgtcgagccagcgggggagggtgcccaccgcggcccctcccgcccagccgcgtgtgcctgtgacccccgcgccggcggtgattcccatcctggtcatcgcctgtgaccgcagcactgttcggcgctgcctggacaagctgctgcattatcggccctcggctgagctcttccccatcatcgttagccaggactgcgggcacgaggagacggcccaggccatcgcctcctacggcagcgcggtcacgcacatccggcagcccgacctgagcagcattgcggtgccgccggaccaccgcaagttccagggctactacaagatcgcgcgccactaccgctgggcgctgggccaggtcttccggcagtttcgcttccccgcggccgtggtggtggaggatgacctggaggtggccccggacttcttcgagtactttcgggccacctatccgctgctgaaggccgacccctccctgtggtgcgtctcggcctggaatgacaacggcaaggagcagatggtggacgccagcaggcctgagctgctctaccgcaccgactttttccctggcctgggctggctgctgttggccgagctctgggctgagctggagcccaagtggccaaaggccttctgggacgactggatgcggcggccggagcagcggcaggggcgggcctgcatacgccctgagatctcaagaacgatgacctttggccgcaagggtgtgagccacgggcagttctttgaccagcacctcaagtttatcaagctgaaccagcagtttgtgcacttcacccagctggacctgtcttacctgcagcgggaggcctatgaccgagatttcctcgcccgcgtctacggtgctccccagctgcaggtggagaaagtgaggaccaatgaccggaaggagctgggggaggtgcgggtgcagtatacgggcagggacagcttcaaggctttcgccaaggctctgggtgtcatggatgaccttaagtcgggggttccgagagctggctaccggggtattgtcaccttccagttccggggccgccgtgtccacctggcgcccccactgacgtgggagggctatgatcctagctggaattagctgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctctcgagaacccttaatcccaagcttacctgctgcgcattgttttatatttgttgtaaaaagtagataattacttccttgatgatctgtaaaaaagagaaaaagaaagcatctaagaacttgaaaaactacgaattagaaaagaccaaatatgtatttcttgcattgaccaatttatgcaagtttatatatatgtaaatgtaagtttcacgaggttctactaaactaaaccacccccttggttagaagaaaagagtgtgtgagaacaggctgttgttgtcacacgattcggacaattctgtttgaaagagagagagtaacagtacgatcgaacgaactttgctctggagatcacagtgggcatcatagcatgtggtactaaaccctttcccgccattccagaaccttcgattgcttgttacaaaacctgtgagccgtcgctaggaccttgttgtgtgacgaaattggaagctgcaatcaataggaagacaggaagtcgagcgtgtctgggttttttcagttttgttctttttgcaaacaaatcacgagcgacggtaatttctttctcgataagaggccacgtgctttatgagggtaacatcaattcaagatctgaattccatgttcgccaacctaaaatacgtttccctgggaattttggtctttcagactaccagtttggttctaacaatgcgttattccagaactttaaaagaagaaggacctcgttatctatcttctacagcagtggttgttgctgaacttttgaagataatggcctgcattttattggtctacaaagacagcaaatgtagtctaagagcactgaatcgagtactacatgatgaaattcttaataaacctatggaaacacttaaacttgctattccatcagggatctatactcttcagaataatttactgtatgtggcactatcaaatctagatgcagctacttatcaggtcacgtatcagttaaaaattcttacaacagcattattttctgtgtctatgcttagtaaaaaattgggtgtataccagtggctgtccctagtaattttgatgacaggagttgcttttgtacagtggccctcagattctcagcttgattctaaggaactttcagctggttctcaatttgtaggactcatggcagttctcacagcatgtttttcaagtggctttgctggggtttactttgagaaaatcttaaaagaaacaaaacaatcagtgtggataagaaatattcagcttggtttctttggaagtatatttggattaatgggtgtatacatttatgatggagaactggtatcaaagaatggattttttcagggatataaccgactgacctggatagtagttgttcttcaggcacttggaggccttgtaatagctgctgttattaagtatgcagataatattttaaaaggatttgcaacctctttatcgataatattatcaacattgatctcctatttttggcttcaagattttgtgccaaccagtgtctttttccttggagccatccttgtaataacagctacttttttgtatggttatgatcccaaacctgcaggaaatcccactaaagcataggtgcggccgcttctttggaattattggaaggtaaggaattgccaggtgttgctttcttatccgaaaagaaataaattgaattgaattgaaatcgatagatcaatttttttcttttctctttccccatcctttacgctaaaataatagtttattttattttttgaatattttttatttatatacgtatatatagactattatttatcttttaatgattattaagatttttattaaaaaaaaattcgctcc

SEQ ID NO: 3 - DNA, 3961 bp, Artificial Sequence
  cassette
taggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaacatgaagttaagtcgccagttcaccgtgtttggcagcgcgatcttctgcgtcgtaatcttctcactctacctgatgctggacaggggtcacttggactaccctcggggcccgcgccaggagggctcctttccgcagggccagctttcaatattgcaagaaaagattgaccatttggagcgtttgctcgctgagaacaacgagatcatctcaaatatcagagactcagtcatcaacctgagcgagtctgtggaggacggcccgcgggggtcaccaggcaacgccagccaaggctccatccacctccactcgccacagttggccctgcaggctgaccccagagactgtttgtttgcttcacagagtgggagtcagccccgggatgtgcagatgttggatgtttacgatctgattccttttgataatccagatggtggagtttggaagcaaggatttgacattaagtatgaagcggatgagtgggaccatgagcccctgcaagtgtttgtggtgcctcactcccataatgacccaggttggttgaagactttcaatgactactttagagacaagactcagtatatttttaataacatggtcctaaagctgaaagaagactcaagcaggaagtttatgtggtctgagatctcttaccttgcaaaatggtgggatattatagatattccgaagaaggaagctgttaaaagtttactacagaatggtcagctggaaattgtgaccggtggctgggttatgcctgatgaagccactccacattattttgccttaattgaccaactaattgaagggcaccaatggctggaaaaaaatctaggagtgaaacctcgatcgggctgggccatagatccctttggtcattcacccacaatggcttatcttctaaagcgtgctggattttcacacatgctcatccagagagtccattatgcaatcaaaaaacacttctctttgcataaaacgctggagtttttctggagacagaattgggatcttggatctgctacagacattttgtgccatatgatgcccttctacagctacgacatccctcacacctgtgggcctgatcctaaaatatgctgccagtttgattttaaacggcttcctggaggcagatatggttgtccctggggagttcccccagaagcaatatctcctggaaatgtccaaagcagggctcagatgctattggatcagtaccggaaaaagtcaaaacttttccgcactaaagttctgctggctccactgggagacgactttcggttcagtgaatacacagagtgggatctgcagtgcaggaactacgagcaactgttcagttacatgaactcgcagcctcatctgaaagtgaagatccagtttggaaccttgtcagattatttcgacgcattggagaaagcggtggcagccgagaagaagagtagccagtctgtgttccctgccctgagtggagacttcttcacgtacgctgacagagacgaccattactggagtggctacttcacgtccagacctttctacaaacgaatggacagaataatggaatctcgtataagggctgctgaaattctttaccagttggccttgaaacaagctcagaaatacaagataaataaatttctttcatcacctcattacacaacactgacagaagccagaaggaacttaggactatttcagcatcatgatgccatcacaggaaccgcgaaagactgggtggttgtggactatggtaccagactctttcagtcattaaattctttggagaagataattggagattctgcatttcttctcattttaaaggacaaaaagctgtaccagtcagatccttccaaagccttcttagagatggatacgaagcaaagttcacaagattctctgccccaaaaaattataatacaactgagcgcacaggagccaaggtaccttgtggtctacaatccctttgaacaagaacggcattcagtggtgtccatccgggtaaactccgccacagtgaaagtgctgtctgattcgggaaaaccggtggaggttcaagtcagtgcagtttggaacgacatgaggacaatttcacaagcagcctatgaggtttcttttctagctcatataccaccactgggactgaaagtgtttaagatcttagagtcacaaagttcaagctcacacttggctgattatgtcctatataataatgatggactagcagaaaatggaatattccacgtgaagaacatggtggatgctggagatgccataacaatagagaatcccttcctggcgatttggtttgaccgatctgggctgatggagaaagtgagaaggaaagaagacagtagacagcatgaactgaaggtccagttcctgtggtacggaaccaccaacaaaagggacaagagcggtgcctacctcttcctgcctgacgggcagggccagccatatgtttccctaagaccgccctttgtcagagtgacacgtggaaggatctactcagatgtgacctgtttcctcgaacacgttactcacaaagtccgcctgtacaacattcagggaatagaaggtcagtccatggaagtttctaatattgtaaacatcaggaatgtgcataaccgtgagattgtaatgagaatttcatctaaaataaacaaccaaaatagatattatactgacctaaatggatatcagattcagcctagaaggaccatgagcaaattgcctcttcaagccaacgtttacccgatgtgcacaatggcgtatatccaggatgctgagcaccggctcacgctgctctctgctcagtctctaggtgcttccagcatggcttctggtcagattgaagtcttcatggatcgaaggctcatgcaggatgataaccgtggccttgggcaaggcgtccatgacaataagattacagctaatttgtttcgaatcctcctcgagaagagaagcgctgtgaacatggaagaagaaaagaagagccctgtcagctacccttccctcctcagccacatgacttcgtccttcctcaaccatccctttctccccatggtactaagtggccagctcccctcccctgcctttgagctgctgagtgaatttcctctgctgcagtcctctctaccttgtgatatccatctggtcaacctgcggacaatacaatcaaagatgggcaaaggctattcggatgaggcagccttgatcctccacaggaaagggtttgattgccagttctccagcagaggcatcgggctaccctgttccactactcagggaaagatgtcagttctgaaacttttcaacaagtttgctgtggagagtctcgtcccttcctctctgtccttgatgcactcccctccagatgcccagaacatgagtgaagtcagcctgagccccatggagatcagcacgttccgtatccgcttgcgttggacctgagatttcggtttctttgaaatttttttgattcggtaatctccgaacagaaggaagaacgaaggaaggagcacagacttagattggtatatatacgcatatgtagtgttgaag

SEQ ID NO: 4 - DNA, 2330 bp, Artificial Sequence
  Sequence of the expression cassette 5
caagcttcctgaaacggagaaacataaacaggcattgctgggatcacccatacatcactctgttttgcctgaccttttccggtaatttgaaaacaaacccggtctcgaagcggagatccggcgataattaccgcagaaataaacccatacacgagacgtagaaccagccgcacatggccggagaaactcctgcgagaatttcgtaaactcgcgcgcattgcatctgtatttcctaatgcggcacttccaggcctcgatcgagaccgtttatccattgcttttttgttgtctttttccctcgttcacagaaagtctgaagaagctatagtagaactatgagctttttttgtttctgttttccttttttttttttttacctctgtggaaattgttactctcacactctttagttcgtttgtttgttttgtttattccaattatgaccggtgacgaaacgtggtcgatggtgggtaccgcttatgctcccctccattagtttcgattatataaaaaggccaaatattgtattattttcaaatgtcctatcattatcgtctaacatctaatttctcttaaattttttctctttctttcctataacaccaatagtgaaaatctttttttcttctatatctacaaaaactttttttttctatcaacctcgttgataaattttttctttaacaatcgttaataattaattaattggaaaataaccattttttctctcttttatacacacattcaaaagaaagaaaaaaaatataccccagcatgtcactttctcttgtatcgtaccgcctaagaaagaacccgtgggttaacaggttccgcatctacaaacggaaggtgctaatcctgacgctcgtggtggccgcctgcggcttcgtcctctggagcagcaatgggcgacaaaggaagaacgaggccctcgccccaccgttgctggacgccgaacccgcgcggggtgccggcggccgcggtggggaccacccctctgtggctgtgggcatccgcagggtctccaacgtgtcggcggcttccctggtcccggcggtcccccagcccgaggcggacaacctgacgctgcggtaccggtccctggtgtaccagctgaactttgatcagaccctgaggaatgtagataaggctggcacctgggccccccgggagctggtgctggtggtccaggtgcataaccggcccgaatacctcagactgctgctggactcacttcgaaaagcccagggaattgacaacgtcctcgtcatctttagccatgacttctggtcgaccgagatcaatcagctgatcgccggggtgaatttctgtccggttctgcaggtgttctttcctttcagcattcagttgtaccctaacgagtttccaggtagtgaccctagagattgtcccagagacctgccgaagaatgccgctttgaaattggggtgcatcaatgctgagtatcccgactccttcggccattatagagaggccaaattctcccagaccaaacatcactggtggtggaagctgcattttgtgtgggaaagagtgaaaattcttcgagattatgctggccttatacttttcctagaagaggatcactacttagccccagacttttaccatgtcttcaaaaagatgtggaaactgaagcagcaagagtgccctgaatgtgatgttctctccctggggacctatagtgccagtcgcagtttctatggcatggctgacaaggtagatgtgaaaacttggaaatccacagagcacaatatgggtctagccttgacccggaatgcctatcagaagctgatcgagtgcacagacactttctgtacttatgatgattataactgggactggactcttcaatacttgactgtatcttgtcttccaaaattctggaaagtgctggttcctcaaattcctaggatctttcatgctggagactgtggtatgcatcacaagaaaacctgtagaccatccactcagagtgcccaaattgagtcactcttaaataataacaaacaatacatgtttccagaaactctaactatcagtgaaaagtttactgtggtagccatttccccacctagaaaaaatggagggtggggagatattagggaccatgaactctgtaaaagttatagaagactgcagtgacgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgct

SEQ ID NO: 5 - DNA, 2452 bp, Artificial Sequence
  Sequence of the expression cassette 6
tgcctgcaggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgactctagaggatccccgggatggccctctttctcagtaagagactgttgagatttaccgtcattgcaggtgcggttattgttctcctcctaacattgaattccaacagtagaactcagcaatatattccgagttccatctccgctgcatttgattttacctcaggatctatatcccctgaacaacaagtcatctctgaggaaaatgatgctaaaaaattagagcaaagtgctctgaattcagaggcaagcgaagactccgaagccccccaactggtcggagtctccacaccgctgcagggcggctcgaacagtgccgccgccatcgggcagtcctccggggagctccggaccggaggggcccggccgccgcctcctctaggcgcctcctcccagccgcgcccgggtggcgactccagcccagtcgtggattctggccctggccccgctagcaacttgacctcggtcccagtgccccacaccaccgcactgtcgctgcccgcctgccctgaggagtccccgctgcttgtgggccccatgctgattgagtttaacatgcctgtggacctggagctcgtggcaaagcagaacccaaatgtgaagatgggcggccgctatgcccccagggactgcgtctctcctcacaaggtggccatcatcattccattccgcaaccggcaggagcacctcaagtactggctatattatttgcacccagtcctgcagcgccagcagctggactatggcatctatgttatcaaccaggcgggagacactatattcaatcgtgctaagctcctcaatgttggctttcaagaagccttgaaggactatgactacacctgctttgtgtttagtgacgtggacctcattccaatgaatgaccataatgcgtacaggtgtttttcacagccacggcacatttccgttgcaatggataagtttggattcagcctaccttatgttcagtattttggaggtgtctctgctctaagtaaacaacagtttctaaccatcaatggatttcctaataattattggggctggggaggagaagatgatgacatttttaacagattagtttttagaggcatgtctatatctcgcccaaatgctgtggtcgggaggtgtcgcatgatccgccactcaagagacaagaaaaatgaacccaatcctcagaggtttgaccgaattgcacacacaaaggagacaatgctctctgatggtttgaactcactcacctaccaggtgctggatgtacagagatacccattgtatacccaaatcacagtggacatcgggacaccgagctaggatcctggtacgttcctcaaggtgctcgtgtctacaccgaaaaattccaatgttctaacgacacctacgtcagatacgtcattaacgatgctgttgttccaattgaaacctgttccactggtccagggttctcttgtgaaatcaatgacttctacgactatgctgaaaagagagtagccggtactgacttcctaaaggtctgtaacgtcagcagcgtcagtaactctactgaattgaccttctactgggactggaacactactcattacaacgccagtctattgagacaatagttttgtataactaaataatattggaaactaaatacgaatacccaaattttttatctaaattttgccgaaagattaaaatctgcag

SEQ ID NO: 6 - DNA, 4917 bp, Artificial Sequence
  Sequence of the expression cassette 7
cagtgtgacgaatatagcgaacaactattgtgtttgaattttaacgtttatctttttatgatttttttaaaaaaacttcctagaaaatttcttatatatctctatttaatgaaaaaccaaagtgatcagaattacaattcatcgtgaatggcatcttcttcgtcagccaattcagcgtcagcatcggcttcctcagcagctttttcctgggcttcttcgtacaaggccttaccgtcgacgtcgaagtgaccgttttccttgatgaagtcgaataaagagtccaaggatcttgaaccttggtacacaacagattcggacttcttaccacctgggtataagacgattgttgggtaaccttcaattacgacgcctctgacatcgttttcagtgtggtctagtttagcaatcaaaacgtcggatgtggcgttggcgtaggtatcagctagttcttggtaagttggggccaatctcttacagtgaccacaccatggggcatagtacaaaacaagaacgtccttctttgggtcgttgacgatttcgtcatggttcttaccgaccaattggaagacagaggaatcttggttctcgaagatctcttgggacttcacgattggggaggcatcacctttcaagaagtccttaaccaaagattcaatagccttagactccaacacgatcttgtcgctcaattcgtcaaacgcctcttcagagagttgaggcaaaccgtacttcaagtcttcagtcatgtcgtggatggcaaatagagggaattgttccttcatgttcaagttgccggcgtgtctgccgaattttctggcatcgatgctaacaaagttcattagacctctgttctttttggccaactcggtaaagagaggcttgtattcttccaattcttcctcgtcattgtagaataagtaacccaaaggcaaaccgctttcgacgtattgggcgaaaacggaaccgtcgatttcaccaaagtagggcaaggcttccacttgcaaccatttttcaaaaacatcagcgtcagcgatatcggctttcttaccgttgtatactacaggctcgtccatggcggagggcaagtaaatagaaagcttgaaatcatcgtctgcgttttcagcggagacaaagtcgtagtcgttgaagtgtttgttggccatggagtaaaaggtggcgttgaagtcggcgtcaatcttaccggattggacgataactggagtgacaaaagtctcgttagcaaggtaagctggtagatcagcaacaacggcgacagccggttggctttgcttgatcatgaattggacaatggcctcggcagttctaggtccctcgtaatcgatcgagttgttaacatcgctgtttttgaaaatcttcaagcttgggaaccctggaatgttgtgttccatacacagatcctggttttcagtacagtcgatctgggccaaggtaatgtttttctcaactaaagtctcggcggctttaacgtattcaggagccatgttcttacagtggccacaccatggagcaaaaaactccgcaagcaccaagtcgtgcgactgaatgtactcattgaaggagtcggtggccaacttaacgacagcggagtcttcaggggccacagcctcttgttgggcgaaaacagaggaggcgagcagcagggaggaccatgacaggacggcaccagcagaaaacttcattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaacccatgtttttcaacagactaagcgctggcaagctgctggtaccactctccgtggtcctgtacgcccttttcgtggtaatattacctttacagaattctttccactcctccaatgttttagttagaggtgccgatgatgtagaaaactacggaactgttatcggtattgacttaggtactacttattcctgtgttgctgtgatgaaaaatggtaagactgaaattcttgctaatgagcaaggtaacagaatcaccccatcttacgtggcattcaccgatgatgaaagattgattggtgatgctgcaaagaaccaagttgctgccaatcctcaaaacaccatcttcgacattaagagattgatcggtttgaaatataacgacagatctgttcagaaggatatcaagcacttgccatttaatgtggttaataaagatgggaagcccgctgtagaagtaagtgtcaaaggagaaaagaaggtttttactccagaagaaatttctggtatgatcttgggtaagatgaaacaaattgccgaagattatttaggcactaaggttacccatgctgtcgttactgttcctgcttatttcaatgacgcgcaaagacaagccaccaaggatgctggtaccatcgctggtttgaacgttttgagaattgttaatgaaccaaccgcagccgccattgcctacggtttggataaatctgataaggaacatcaaattattgtttatgatttgggtggtggtactttcgatgtctctctattgtctattgaaaacggtgttttcgaagtccaagccacttctggtgatactcatttaggtggtgaagattttgactataagatcgttcgtcaattgataaaagctttcaagaagaagcatggtattgatgtgtctgacaacaacaaggccctagctaaattgaagagagaagctgaaaaggctaaacgtgccttgtccagccaaatgtccacccgtattgaaattgactccttcgttgatggtatcgacttaagtgaaaccttgaccagagctaagtttgaggaattaaacctagatctattcaagaagaccttgaagcctgtcgagaaggttttgcaagattctggtttggaaaagaaggatgttgatgatatcgttttggttggtggttctactagaattccaaaggtccaacaattgttagaatcatactttgatggtaagaaggcctccaagggtattaacccagatgaagctgttgcatacggtgcagccgttcaagctggtgtcttatccggtgaagaaggtgtcgaagatattgttttattggatgtcaacgctttgactcttggtattgaaaccactggtggtgtcatgactccattaattaagagaaatactgctattcctacaaagaaatcccaaattttctctactgccgttgacaaccaaccaaccgttatgatcaaggtatacgagggtgaaagagccatgtctaaggacaacaatctattaggtaagtttgaattaaccggcattccaccagcaccaagaggtgtacctcaaattgaagtcacatttgcacttgacgctaatggtattctgaaggtgtctgccacagataagggaactggtaaatccgaatctatcaccatcactaacgataaaggtagattaacccaagaagagattgatagaatggttgaagaggctgaaaaattcgcttctgaagacgcttctatcaaggccaaggttgaatctagaaacaaattagaaaactacgctcactctttgaaaaaccaagttaatggtgacctaggtgaaaaattggaagaagaagacaaggaaaccttattagatgctgctaacgatgttttagaatggttagatgataactttgaaaccgccattgctgaagactttgatgaaaagttcgaatctttgtccaaggtcgcttatccaattacttctaagttgtacggaggtgctgatggttctggtgccgctgattatgacgacgaagatgaagatgacgatggtgattatttcgaacacgacgaattgtagataaaatagttaaaaatttttgctgctggaagcttcaaggttgttaatttattgacttgcatagaatatctacatttcttctaaaaatacatgcatagctaattcaaacttcgagcttcatacaattttcgaggagattatactgagtatatacgtaaatatatgcattatatgttataaaattagaaagatatagaaatttcattgaagagtatagagactggggttaaggtactcagtaacagtgtcatcaatatgctaattttgcgtattacttagctctattgcgcaaatgcaattttttcttaccctgataatgctttatttcccgttccgaaaatttttcactgaaaaaaaagtgcttaagctcatctcatctcatctcatcccatcactattgaaatattttgctaaaacattataacagagagagttgaaaggctcgagaacctaatactgaaatggccaaaaaaaatagattgaacacaactcaaagaaaga

SEQ ID NO: 7 - DNA, 16419 bp, Artificial Sequence
  Sequence of pGLY-yac_MCS
ttctcatgtttgacagcttatcatcgatggccatgcaggccgtttaaacggtggccggcactagtgctcgtgcgcatttaaatggtggccggcgcgatcgcgctcgtgcgctcgcgaggtggccggcggccggccgactcggccgaattccgtaatcttgagatcgggcgttcgatcgccccgggagatttttttgttttttatgtcttccattcacttcccagacttgcaagttgaaatatttctttcaagggaattgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgacggtggccggcaattccacttgcaattacataaaaaattccggcggtttttcgcgtgtgactcaatgtcgaaatacctgcctaatgaacatgaacatcgcccaaatgtatttgaagacccgctgggagaagttcaagatatataagtaacaagcagccaatagtataaaaaaaaatctgagtttattacctttcctggaatttcagtgaaaaactgctaattatagagagatatcacagagttactcactaatgactaacgaaaaggtctggatagagaagttggataatccaactctttcagtgttaccacatgactttttacgcccacaacaagaaccttatacgaaacaagctacatattcgttacagctacctcagctcgatgtgcctcatgatagtttttctaacaaatacgctgtcgctttgagtgtatgggctgcattgatatatagagtaaccggtgacgatgatattgttctttatattgcgaataacaaaatcttaagattcaatattcaaccaacgtggtcatttaatgagctgtattctacaattaacaatgagttgaacaagctcaattctattgaggccaatttttcctttgacgagctagctgaaaaaattcaaagttgccaagatctggaaaggacccctcagttgttccgtttggcctttttggaaaaccaagatttcaaattagacgagttcaagcatcatttagtggactttgctttgaatttggataccagtaataatgcgcatgttttgaacttaatttataacagcttactgtattcgaatgaaagagtaaccattgttgcggaccaatttactcaatatttgactgctgcgctaagcgatccatccaattgcataactaaaatctctctgatcaccgcatcatccaaggatagtttacctgatccaactaagaacttgggctggtgcgatttcgtggggtgtattcacgacattttccaggacaatgctgaagccttcccagagagaacctgtgttgtggagactccaacactaaattccgacaagtcccgttctttcacttatcgcgacatcaaccgcacttctaacatagttgcccattatttgattaaaacaggtatcaaaagaggtgatgtagtgatgatctattcttctaggggtgtggatttgatggtatgtgtgatgggtgtcttgaaagccggcgcaaccttttcagttatcgaccctgcatatcccccagccagacaaaccatttacttaggtgttgctaaaccacgtgggttgattgttattagagctgctggacaattggatcaactagtagaagattacatcaatgatgaattggagattgtttcaagaatcaattccatcgctattcaagaaaatggtaccattgaaggtggcaaattggacaatggcgaggatgttttggctccatatgatcactacaaagacaccagaacaggtgttgtagttggaccagattccaacccaaccctatctttcacatctggttccgaaggtattcctaagggtgttcttggtagacatttttccttggcttattatttcaattggatgtccaaaaggttcaacttaacagaaaatgataaattcacaatgctgagcggtattgcacatgatccaattcaaagagatatgtttacaccattatttttaggtgcccaattgtatgtccctactcaagatgatattggtacaccgggccgtttagcggaatggatgagtaagtatggttgcacagttacccatttaacacctgccatgggtcaattacttactgcccaagctactacaccattccctaagttacatcatgcgttctttgtgggtgacattttaacaaaacgtgattgtctgaggttacaaaccttggcagaaaattgccgtattgttaatatgtacggtaccactgaaacacagcgtgcagtttcttatttcgaagttaaatcaaaaaatgacgatccaaactttttgaaaaaattgaaagatgtcatgcctgctggtaaaggtatgttgaacgttcagctactagttgttaacaggaacgatcgtactcaaatatgtggtattggcgaaataggtgagatttatgttcgtgcaggtggtttggccgaaggttatagaggattaccagaattgaataaagaaaaatttgtgaacaactggtttgttgaaaaagatcactggaattatttggataaggataatggtgaaccttggagacaattctggttaggtccaagagatagattgtacagaacgggtgatttaggtcgttatctaccaaacggtgactgtgaatgttgcggtagggctgatgatcaagttaaaattcgtgggttcagaatcgaattaggagaaatagatacgcacatttcccaacatccattggtaagagaaaacattactttagttcgcaaaaatgccgacaatgagccaacattgatcacatttatggtcccaagatttgacaagccagatgacttgtctaagttccaaagtgatgttccaaaggaggttgaaactgaccctatagttaagggcttaatcggttaccatcttttatccaaggacatcaggactttcttaaagaaaagattggctagctatgctatgccttccttgattgtggttatggataaactaccattgaatccaaatggtaaagttgataagcctaaacttcaattcccaactcccaagcaattaaatttggtagctgaaaatacagtttctgaaactgacgactctcagtttaccaatgttgagcgcgaggttagagacttatggttaagtatattacctaccaagccagcatctgtatcaccagatgattcgtttttcgatttaggtggtcattctatcttggctaccaaaatgatttttaccttaaagaaaaagctgcaagttgatttaccattgggcacaattttcaagtatccaacgataaaggcctttgccgcggaaattgacagaattaaatcatcgggtggatcatctcaaggtgaggtcgtcgaaaatgtcactgcaaattatgcggaagacgccaagaaattggttgagacgctaccaagttcgtacccctctcgagaatattttgttgaacctaatagtgccgaaggaaaaacaacaattaatgtgtttgttaccggtgtcacaggatttctgggctcctacatccttgcagatttgttaggacgttctccaaagaactacagtttcaaagtgtttgcccacgtcagggccaaggatgaagaagctgcatttgcaagattacaaaaggcaggtatcacctatggtacttggaacgaaaaatttgcctcaaatattaaagttgtattaggcgatttatctaaaagccaatttggtctttcagatgagaagtggatggatttggcaaacacagttgatataattatccataatggtgcgttagttcactgggtttatccatatgccaaattgagggatccaaatgttatttcaactatcaatgttatgagcttagccgccgtcggcaagccaaagttctttgactttgtttcctccacttctactcttgacactgaatactactttaatttgtcagataaacttgttagcgaagggaagccaggcattttagaatcagacgatttaatgaactctgcaagcgggctcactggtggatatggtcagtccaaatgggctgctgagtacatcattagacgtgcaggtgaaaggggcctacgtgggtgtattgtcagaccaggttacgtaacaggtgcctctgccaatggttcttcaaacacagatgatttcttattgagatttttgaaaggttcagtccaattaggtaagattccagatatcgaaaattccgtgaatatggttccagtagatcatgttgctcgtgttgttgttgctacgtctttgaatcctcccaaagaaaatgaattggccgttgctcaagtaacgggtcacccaagaatattattcaaagactacttgtatactttacacgattatggttacgatgtcgaaatcgaaagctattctaaatggaagaaatcattggaggcgtctgttattgacaggaatgaagaaaatgcgttgtatcctttgctacacatggtcttagacaacttacctgaaagtaccaaagctccggaactagacgataggaacgccgtggcatctttaaagaaagacaccgcatggacaggtgttgattggtctaatggaataggtgttactccagaagaggttggtatatatattgcatttttaaacaaggttggatttttacctccaccaactcataatgacaaacttccactgccaagtatagaactaactcaagcgcaaataagtctagttgcttcaggtgctggtgctcgtggaagctccgcagcagcttaaggttgagcattacgtatgatatgtccatgtacaataattaaatatgaattaggagaaagacttagcttcttttcgggtgatgtcacttaaaaactccgagaataatatataataagagaataaaatattagttattgaataagaactgtaaatcagctggcgttagtctgctaatggcagcttcatcttggtttattgtagcatgaatcatatttgcctttttttcctgtaattcaatgattcttgcttctatactatcctcaatgcaaaaccttgtgatcttcacaggtcgatactgaccaattctatgaactctatcaccactttgccattcaacactagggttccaccatgggtctaaaatgaatacttgcgaagctgcggccgccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagggcggtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgcgcagaactcgtggtgctgggcactgctgctgctgcggcagctggcaacctgacttgtatcgtcgcgatcggaaatgagaacaggggcatcttgagccctgcggacggtgccgacaggttcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtcccgggagatctgcatgtctactaaactcacaaattagagcttcaatttaattatatcagttattaccctccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagccctcgagggataagcttcatttttagataaaatttattaatcatcattaatttcttgaaaaacattttatttattgatcttttataacaaaaaacccttctaaaagtttatttttgaatgaaaaacttataaaaatttatgaaaactacaaaaaataaaatttttaattaaaataattttgataagaacttcaatctttgactagctagcttagtcatttttgagatttaattaatattttatgtttattcatatataaactattcaaaatattatagaatttaaacattttaacatcttaatcattcataaataactaaaaatcaaagtattacatcaataaataacttttactcaatgtcaaagaattattggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggtgggaaaacagcattcaggtattagaagaatatcctgattcaggtgaaaatattgttgatgcgcgggatccgagctcggctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccccccccccttttctttccaattttttttttttcgtcattataaaaatcattacgcccgagtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaagggggggggggatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtgagctcggatcccgcgcatcaacaatattttcacctgaatcaggatattcttctaatacctgaatgctgttttcccaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaataattctttgacattgagtaaaagttatttattgatgtaatactttgatttttagttatttatgaatgattaagatgttaaaatgtttaaattctataatattttgaatagtttatatatgaataaacataaaatattaattaaatctcaaaaatgactaagctagctagtcaaagattgaagttcttatcaaaattattttaattaaaaattttattttttgtagttttcataaatttttataagtttttcattcaaaaataaacttttagaagggttttttgttataaaagatcaataaataaaatgtttttcaagaaattaatgatgattaataaattttatctaaaaatgaagcttatccctcgagggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcaaaaacttatcgaaagatgacgactttttcttaattctcgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgatcgagaaaaaaaatataaaagagatggaggaacgggaaaaagttagttgtggtgataggtggcaagtggtattccgtaagaacaacaagaaaagcatttcatattatggctgaactgagcgaacaagtgcaaaatttaagcatcaacgacaacaacgagaatggttatgttcctcctcacttaagaggaaaaccaagaagtgccagaaataacatgagcaactacaataacaacaacggcggctacaacggtggccgtggcggtggcagcttctttagcaacaaccgtcgtggtggttacggcaacggtggacgtctaagaaaccattattatcatgacattaacctataaaaataggcgtatcacgaggccctttcgtcttcaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcaggccttttgaaaagcaagcataaaagatctaaacataaaatctgtaaaataacaagatgtaaagataatgctaaatcatttggctttttgattgattgtacaggaaaatatacatcgcagggggttgacttttaccatttcaccgcaatggaatcaaacttgttgaagagaatgttcacaggcgcatacgctacaatgacccgattcttgctagccttttctcggtcttgcaaacaaccgccggcagcttagtatataaatacacatgtacatacctctctccgtatcctcgtaatcattttcttgtatttatcgtcttttcgctgtaaaaactttatcacacttatctcaaatacacttattaaccgcttttactattatcttctacgctgacagtaatatcaaacagtgacacatattaaacacagtggtttctttgcataaacaccatcagcctcaagtcgtcaagtaaagatttcgtgttcatgcagatagataacaatctatatgttgataattagcgttgcctcatcaatgcgagatccgtttaaccggaccctagtgcacttaccccacgttcggtccactgtgtgccgaacatgctccttcactattttaacatgtggaattaattctaaatcctctttatatgatctgccgatagatagttctaagtcattgaggttcatcaacaattggattttctgtttactcgacttcaggtaaatgaaatgagatgatacttgcttatctcatagttaactctaagaggtgatacttatttactgtaaaactgtgacgataaaaccggaaggaagaataagaaaactcgaactgatctataatgcctattttctgtaaagagtttaagctatgaaagcctcggcattttggccgctcctaggtagtgctttttttccaaggacaaaacagtttctttttcttgagcaggttttatgtttcggtaatcataaacaataaataaattatttcatttatgtttaaaaataaaaaataaaaaagtattttaaatttttaaaaaagttgattataagcatgtgaccttttgcaagcaattaaattttgcaatttgtgattttaggcaaaagttacaatttctggctcgtgtaatatatgtatgctaaagtgaacttttacaaagtcgatatggacttagtcaaaagaaattttcttaaaaatatatagcactagccaatttagcacttctttatgagatatattatagactttattaagccagatttgtgtattatatgtatttacccggcgaatcatggacatacattctgaaataggtaatattctctatggtgagacagcatagataacctaggatacaagttaaaagctagtactgttttgcagtaatttttttcttttttataagaatgttaccacctaaataagttataaagtcaatagttaagtttgatatttgattgtaaaataccgtaatatatttgcatgatcaaaaggctcaatgttgactagccagcatgtcaaccactatattgatcaccgatatatggacttccacaccaactagtaatatgacaataaattcaagatattcttcatgagaatggcccaga

SEQ ID NO: 8 - DNA, 397 bp, Artificial Sequence
  Promotor TEF
taggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaac

SEQ ID NO: 9 - DNA, 1029 bp, Artificial Sequence
  ORF hph (hygromycine)
atgggtaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataa

SEQ ID NO: 10 - DNA, 249 bp, Artificial Sequence
  Terminator PGK
gtgcggccgcttctttggaattattggaaggtaaggaattgccaggtgttgctttcttatccgaaaagaaataaattgaattgaattgaaatcgatagatcaatttttttcttttctctttccccatcctttacgctaaaataatagtttattttattttttgaatattttttatttatatacgtatatatagactattatttatcttttaatgattattaagatttttattaaaaaaaaattcgctcc

SEQ ID NO: 11 - DNA, 40 bp, Artificial Sequence
  Mnn1 5′ end primer
tttatattaaaccaaaggtctttgagatcgtgtaccatac

SEQ ID NO: 12 - DNA, 40 bp, Artificial Sequence
  Mnn1 3′ end primer
tgtcaacactgtttggtcacatgctaataaagataacacc

SEQ ID NO: 13 - DNA, 13 bp, Artificial Sequence
  SfiI restriction site
ggccatgcaggcc

SEQ ID NO: 14 - DNA, 13 bp, Artificial Sequence
  SfiI restriction site
ggcccgtacggcc

SEQ ID NO: 15 - DNA, 13 bp, artificial
  SfiI restriction site
ggcctgacgggcc

SEQ ID NO: 16 - DNA, 13 bp, Artificial Sequence
  SfiI restriction site
ggccgctatggcc

SEQ ID NO: 17 - DNA, 13 bp, artificial
  SfiI restriction site
ggccacgctggcc

SEQ ID NO: 18 - DNA, 13 bp, Artificial Sequence
  SfiI restriction site
ggcccctgaggcc

SEQ ID NO: 19 - DNA, 13 bp, Artificial Sequence
  SfiI restriction site
ggccgactcggcc

SEQ ID NO: 20 - DNA, 16584 bp, Artificial Sequence
  Sequence of the pGLY-yac-hph_MCS
atggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgcgcagaactcgtggtgctgggcactgctgctgctgcggcagctggcaacctgacttgtatcgtcgcgatcggaaatgagaacaggggcatcttgagcccctgcggacggtgccgacaggttcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtcccgggagatctgcatgtctactaaactcacaaattagagcttcaatttaattatatcagttattaccctccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagccctcgagggataagcttcatttttagataaaatttattaatcatcattaatttcttgaaaaacattttatttattgatcttttataacaaaaaacccttctaaaagtttatttttgaatgaaaaacttataaaaatttatgaaaactacaaaaaataaaatttttaattaaaataattttgataagaacttcaatctttgactagctagcttagtcatttttgagatttaattaatattttatgtttattcatatataaactattcaaaatattatagaatttaaacattttaacatcttaatcattcataaataactaaaaatcaaagtattacatcaataaataacttttactcaatgtcaaagaattattggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggttggggtgggaaaacagcattcaggtattagaagaatatcctgattcaggtgaaaatattgttgatgcgcgggatccgagctcggctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcggggggggggggggggttttctttccaattttttttttttcgtcattataaaaatcattacgaccgagattcccggccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaggaaaccgaaatcaaaaaaaagaataaaaaaaaaatgatgaattgaaacccccccccccccccccgatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtgagctcggatcccgcgcatcaacaatattttcacctgaatcaggatattcttctaatacctgaatgctgttttcccaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaaccccaataattctttgacattgagtaaaagttatttattgatgtaatactttgatttttagttatttatgaatgattaagatgttaaaatgtttaaattctataatattttgaatagtttatatatgaataaacataaaatattaattaaatctcaaaaatgactaagctagctagtcaaagattgaagttcttatcaaaattattttaattaaaaattttattttttgtagttttcataaatttttataagtttttcattcaaaaataaacttttagaagggttttttgttataaaagatcaataaataaaatgtttttcaagaaattaatgatgattaataaattttatctaaaaatgaagcttatccctcgagggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaggtctagagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaacatgggtaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataagtgcggccgcttctttggaattattggaaggtaaggaattgccaggtgttgctttcttatccgaaaagaaataaattgaattgaattgaaatcgatagatcaatttttttcttttctctttccccatcctttacgctaaaataatagtttattttattttttgaatattttttatttatatacgtatatatagactattatttatcttttaatgattattaagatttttattaaaaaaaaattcgctccgacgtctaagaaaccattattatcatgacattaacctataaaaataggcgtatcacgaggccctttcgtcttcaagaaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcaggccttttgaaaagcaagcataaaagatctaaacataaaatctgtaaaataacaagatgtaaagataatgctaaatcatttggctttttgattgattgtacaggaaaatatacatcgcagggggttgacttttaccatttcaccgcaatggaatcaaacttgttgaagagaatgttcacaggcgcatacgctacaatgacccgattcttgctagccttttctcggtcttgcaaacaaccgccggcagcttagtatataaatacacatgtacatacctctctccgtatcctcgtaatcattttcttgtatttatcgtcttttcgctgtaaaaactttatcacacttatctcaaatacacttattaaccgcttttactattatcttctacgctgacagtaatatcaaacagtgacacatattaaacacagtggtttctttgcataaacaccatcagcctcaagtcgtcaagtaaagatttcgtgttcatgcagatagataacaatctatatgttgataattagcgttgcctcatcaatgcgagatccgtttaaccggaccctagtgcacttaccccacgttcggtccactgtgtgccgaacatgctccttcactattttaacatgtggaattaattctaaatcctctttatatgatctgccgatagatagttctaagtcattgaggttcatcaacaattggattttctgtttactcgacttcaggtaaatgaaatgagatgatacttgcttatctcatagttaactctaagaggtgatacttatttactgtaaaactgtgacgataaaaccggaaggaagaataagaaaactcgaactgatctataatgcctattttctgtaaagagtttaagctatgaaagcctcggcattttggccgctcctaggtagtgctttttttccaaggacaaaacagtttctttttcttgagcaggttttatgtttcggtaatcataaacaataaataaattatttcatttatgtttaaaaataaaaaataaaaaagtattttaaatttttaaaaaagttgattataagcatgtgaccttttgcaagcaattaaattttgcaatttgtgattttaggcaaaagttactatttctggctcgtgtaatatatgtatgctaatgtgaacttttacaaagtcgatatggacttagtcaaaagaaattttcttaaaaatatatagcactagccaatttagcacttctttatgagatatattatagactttattaagccagatttgtgtattatatgtatttacccggcgaatcatggacatacattctgaaataggtaatattctctatggtgagacagcatagataacctaggatacaagttaaaagctagtactgttttgcagtaatttttttcttttttataagaatgttaccacctaaataagttataaagtcaatagttaagtttgatatttgattgtaaaataccgtaatatatttgcatgatcaaaaggctcaatgttgactagccagcatgtcaaccactatattgatcaccgatattaggacttccacaccaactagtaatatgacaataaattcaagatattcttcatgagaatggcccagctcatgtttgacagcttatcatcgatggccatgcaggccgtttaaacggtggccggcactagtgctcgtgcgcatttaaatggtggccggcgcgatcgcgctcgtgcgctcgcgaggtggccggcggccggccgactcggccgaattccgtaatcttgagatcgggcgttcgactcgcccccgggagatttttttgttttttatgtctccattcacttcccagacttgcaagttgaaatatttctttcaagaattgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgacggtggccggcaattccacttgcaattacataaaaaattccggcggtttttcgcgtgtgactcaatgtcgaaatacctgcctaatgaacatgaacatcgcccaaatgtatttgaagacccgctgggagaagttcaagatatataagtaacaagcagccaatagtataaaaaaaaatctgagtttattacctttcctggaatttcagtgaaaaactgctaattatagagagatatcacagagttactcactaatgactaacgaaaaggtctggatagagaagttggataatccaactctttcagtgttaccacatgactttttacgcccacaacaagaaccttatacgaaacaagctacatattcgttacagctacctcagctcgatgtgcctcatgatagtttttctaacaaatacgctgtcgctttgagtgtatgggctgcattgatatatagagtaaccggtgacgatgatattgttctttatattgcgaataacaaaatcttaagattcaatattcaaccaacgtggtcatttaatgagctgtattctacaattaacaatgagttgaacaagctcaattctattgaggccaatttttcctttgacgagctagctgaaaaaattcaaagttgccaagatctggaaaggacccctcagttgttccgtttggcctttttggaaaaccaagatttcaaattagacgagttcaagcatcatttagtggactttgctttgaatttggataccagtaataatgcgcatgttttgaacttaatttataacagcttactgtattcgaatgaaagagtaaccattgttgcggaccaatttactcaatatttgactgctgcgctaagcgatccatccaattgcataactaaaatctctctgatcaccgcatcatccaaggatagtttacctgatccaactaagaacttgggctggtgcgatttcgtggggtgtattcacgacattttccaggacaatgctgaagccttcccagagagaacctgtgttgtggagactccaacactaaattccgacaagtcccgttctttcacttatcgcgacatcaaccgcacttctaacatagttgcccattatttgattaaaacaggtatcaaaagaggtgatgtagtgatgatctattcttctaggggtgtggatttgatggtatgtgtgatgggtgtcttgaaagccggcgcaaccttttcagttatcgaccctgcatatcccccagccagacaaaccatttacttaggtgttgctaaaccacgtgggttgattgttattagagctgctggacaattggatcaactagtagaagattacatcaatgatgaattggagattgtttcaagaatcaattccatcgctattcaagaaaatggtaccattgaaggtggcaaattggacaatggcgaggatgttttggctccatatgatcactacaaagacaccagaacaggtgttgtagttggaccagattccaacccaaccctatctttcacatctggttccgaaggtattcctaagggtgttcttggtagacatttttccttggcttattatttcaattggatgtccaaaaggttcaacttaacagaaaatgataaattcacaatgctgagcggtattgcacatgatccaattcaaagagatatgtttacaccattatttttaggtgcccaattgtatgtccctactcaagatgatattggtacaccgggccgtttagcggaatggatgagtaagtatggttgcacagttacccatttaacacctgccatgggtcaattacttactgcccaagctactacaccattccctaagttacatcatgcgttctttgtgggtgacattttaacaaaacgtgattgtctgaggttacaaaccttggcagaaaattgccgtattgttaatatgtacggtaccactgaaacacagcgtgcagtttcttatttcgaagttaaatcaaaaaatgacgatccaaactttttgaaaaaattgaaagatgtcatgcctgctggtaaaggtatgttgaacgttcagctactagttgttaacaggaacgatcgtactcaaatatgtggtattggcgaaataggtgagatttatgttcgtgcaggtggtttggccgaaggttatagaggattaccagaattgaataaagaaaaatttgtgaacaactggtttgttgaaaaagatcactggaattatttggataaggataatggtgaaccttggagacaattctggttaggtccaagagatagattgtacagaacgggtgatttaggtcgttatctaccaaacggtgactgtgaatgttgcggtagggctgatgatcaagttaaaattcgtgggttcagaatcgaattaggagaaatagatacgcacatttcccaacatccattggtaagagaaaacattactttagttcgcaaaaatgccgacaatgagccaacattgatcacatttatggtcccaagatttgacaagccagatgacttgtctaagttccaaagtgatgttccaaaggaggttgaaactgaccctatagttaagggcttaatcggttaccatcttttatccaaggacatcaggactttcttaaagaaaagattggctagctatgctatgccttccttgattgtggttatggataaactaccattgaatccaaatggtaaagttgataagcctaaacttcaattcccaactcccaagcaattaaatttggtagctgaaaatacagtttctgaaactgacgactctcagtttaccaatgttgagcgcgaggttagagacttatggttaagtatattacctaccaagccagcatctgtatcaccagatgattcgtttttcgatttaggtggtcattctatcttggctaccaaaatgatttttaccttaaagaaaaagctgcaagttgatttaccattgggcacaattttcaagtatccaacgataaaggcctttgccgcggaaattgacagaattaaatcatcgggtggatcatctcaaggtgaggtcgtcgaaaatgtcactgcaaattatgcggaagacgccaagaaattggttgagacgctaccaagtttgtacccctctcgagaatattttgttgaacctaatagtgccgaaggaaaaacaacaattaatgtgtttgttaccggtgtcacaggatttctgggctcctacatccttgcagatttgttaggacgttctccaaagaactacagtttcaaagtgtttgcccacgtcagggccaaggatgaagaagctgcatttgcaagattacaaaaggcaggtatcacctatggtacttggaacgaaaaatttgcctcaaatattaaagttgtattaggcgatttatctaaaagccaatttggtctttcagatgagaagtggatggatttggcaaacacagttgatataattatccataatggtgcgttagttcactgggtttatccatatgccaaattgagggatccaaatgttatttcaactatcaatgttatgagcttagccgccgtcggcaagccaaagttctttgactttgtttcctccacttctactcttgacactgaatactactttaatttgtcagataaacttgttagcgaagggaagccaggcattttagaatcagacgatttaatgaactctgcaagcgggctcactggtggatatggtcagtccaaatgggctgctgagtacatcattagacgtgcaggtgaaaggggcctacgtgggtgtattgtcagaccaggttacgtaacaggtgcctctgccaatggttcttcaaacacagatgatttcttattgagatttttgaaaggttcagtccaattaggtaagattccagatatcgaaaattccgtgaatatggttccagtagatcatgttgctcgtgttgttgttgctacgtctttgaatcctcccaaagaaaatgaattggccgttgctcaagtaacgggtcacccaagaatattattcaaagactacttgtatactttacacgattatggttacgatgtcgaaatcgaaagctattctaaatggaagaaatcattggaggcgtctgttattgacaggaatgaagaaaatgcgttgtatcctttgctacacatggtcttagacaacttacctgaaagtaccaaagctccggaactagacgataggaacgccgtggcatctttaaagaaagacaccgcatggacaggtgttgattggtctaatggaataggtgttactccagaagaggttggtatatatattgcatttttaaacaaggttggatttttacctccaccaactcataatgacaaacttccactgccaagtatagaactaactcaagcgcaaataagtctagttgcttcaggtgctggtgctcgtggaagctccgcagcagcttaaggttgagcattacgtatgatatgtccatgtacaataattaaatatgaattaggagaaagacttagcttcttttcgggtgatgtcacttaaaaactccgagaataatatataataagagaataaaatattagttattgaataagaactgtaaatcagctggcgttagtctgctaatggcagcttcatcttggtttattgtagcatgaatcatatttgcctttttttcctgtaattcaatgattcttgcttctatactatcctcaatgcaaaaccttgtgatcttcacaggtcgatactgaccaattctatgaactctatcaccactttgccattcaacactagggttccaccatgggtctaaaatgaatacttgcgaagctgcggccgccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagggcggtgttgacaattaatcatcggcatagtatatcggcatagtataatacgacaaggtgaggaactaaacc

SEQ ID NO: 21 - DNA, 5935 bp, Artificial Sequence
  Sequence of the expression cassette 8
gctcattttttagtatattcttcgaagaaatcacattactttatataatgtataattcattatgtgataatgccaatcgctaagaaaaaaaaagagtcatccgctaggtggaaaaaaaaaaatgaaaatcattaccgaggcataaaaaaatatagagtgtactagaggaggccaagagtaatagaaaaagaaaattgcgggaaaggactgtgttatgcaaaacaacaacgaatttaaaattggtaatcgttcagtaggttacaaccacgaaccattgattatctgtgaaatcggcatcaatcatgaaggctctttaaaaacagcttttgaaatggttgatgctgcctataatgcaggcgctgaagttgttaaacatcaaacacacatcgttgaagacgaaatgtctgatgaggccaaacaagtcattccaggcaatgcagatgtctctatttatgaaattatggaacgttgcgccctgaatgaagaagatgagattaaattaaaagaatacgtagagagtaagggtatgatttttatcagtactcctttctctcgtgcagctgctttacgattacaacgtatggatattccagcatataaaatcggctctggcgaatgtaataactacccattaattaaactggtggcctcttttggtaagcctattattctctctaccggcatgaattctattgaaagcatcaaaaagtcggtagaaattattcgagaagcaggggtaccttatgctttgcttcactgtaccaacatctacccaaccccttacgaagatgttcgattgggtggtatgaacgatttatctgaagcctttccagacgcaatcattggcctgtctgaccataccttagataactatgcttgcttaggagcagtagctttaggcggttcgattttagagcgtcactttactgaccgcatggatcgcccaggtccggatattgtatgctctatgaatccggatacttttaaagagctcaagcaaggcgctcatgctttaaaattggcacgcggcggcaaaaaagacacgattatcgcgggagaaaagccaactaaagatttcgcctttgcatctgtcgtagcagataaagacattaaaaaaggagaactgttgtccggagataacctatgggttaaacgcccaggcaatggagacttcagcgtcaacgaatatgaaacattatttggtaaggtcgctgcttgcaatattcgcaaaggtgctcaaatcaaaaaaactgatattgaataagattaatataattatataaaaatattatcttcttttctttatatctagtgttatgtaaaataaattgatgactacggaaagcttttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacagaagagtcgacgcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctgtcgacatggaaaaacaaaatattgcggttatacttgcgcgccaaaactccaaaggattgccattaaaaaatctccggaaaatgaatggcatatcattacttggtcatacaattaatgctgctatatcatcaaagtgttttgaccgcataattgtttcgactgatggcgggttaattgcagaagaagctaaaaatttcggtgtcgaagtcgtcctacgccctgcagagctggcctccgatacagccagctctatttcaggtgtaatacatgctttagaaacaattggcagtaattccggcacagtaaccctattacaaccaaccagtccattacgcacaggggctcatattcgtgaagctttttctctatttgatgagaaaataaaaggatccgttgtctctgcatgcccaatggagcatcatccactaaaaaccctgcttcaaatcaataatggcgaatatgcccccatgcgccatctaagcgatttggagcagcctcgccaacaattacctcaggcatttaggcctaatggtgcaatttacattaatgatactgcttcactaattgcaaataattgtttttttatcgctccaaccaaactttatattatgtctcatcaagactctatcgatattgatactgagcttgatttacaacaggcagaaaacattcttaatcacaaggaaagctaaaaaactgtattataagtaaatgcatgtatactaaactcacaaattagagcttcaatttaattatatcagttattacccgatatccttacccataaggttgtttgtgacggcgtcgtacaagagaacgtgggaactttttaggctcaccaaaaaagaaagaaaaaatacgagttgctgacagaagcctcaagaaaaaaaaaattcttcttcgactatgctggaggcagagatgatcgagccggtagttaactatatatagctaaattggttccatcaccttcttttctggtgtcgctccttctagtgctatttctggcttttcctatttttttttttccatttttctttctctctttctaatatataaattctcttgcattttctatttttctctctatctattctacttgtttattcccttcaaggtttttttttaaggagtacttgtttttagaatatacggtcaacgaactataattaactaaacgatatcatggctccggcgagagaaaatgtcagtttattcttcaagctgtactgcttggcggtgatgactctggtggctgccgcttacaccgtagctttaagatacacaaggacaacagctgaagaactctacttctcaaccactgccgtgtgtatcacagaagtgataaagttactgataagtgttggcctgttagctaaggaaactggcagtttgggtagatttaaagcctcattaagtgaaaatgtcttggggagccccaaggaactggcgaagttgagtgtgccatcactagtgtatgctgtgcagaacaacatggccttcctggctctcagtaatctggatgcagcagtgtaccaggtgacctatcaactgaagatcccctgcactgctttatgtactgttttaatgttaaatcgaacactcagcaaattacagtggatttccgtcttcatgctgtgtggtggggtcacactcgtacagtggaaaccagcccaagcttcaaaagtcgtggtagcgcagaatccattgttaggctttggtgctatagctattgctgtattgtgctctggatttgcaggagtttattttgaaaaagtcttaaagagttccgacacttccctttgggtgagaaacattcagatgtatctgtcagggatcgttgtgacgttagctggtacctacttgtcagatggagctgaaattcaagaaaaaggattcttctatggctacacgtattatgtctggtttgttatcttccttgctagtgtgggaggcctctacacgtcagtggtggtgaagtatacagacaacatcatgaaaggcttctctgctgccgcagccattgttctttctaccattgcttcagtcctactgtttggattacagataacactttcatttgcactgggagctcttcttgtgtgtgtttccatatatctctatgggttacccagacaagatactacatccattcaacaagaagcaacttcaaaagagagaatcattggtgtgtgagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgaccgatcgaaccggccggacagtaatatagtaatcgttttgtacgtttttcaagaagcgacgcacaactgttttccattttttttttttttttttcagtgatcatcgtccatgaaaaaaatttttcatttgtctctttcgtgcttcctggatatataaaatacgatttatttagttgtctttgtcaatcctcatctttctttactcattatttcatttcggttttgtcatctctagaacaacacagttactacaacaatcaatccgatcgatggccctctttctcagtaagagactgttgagatttaccgtcattgcaggtgcggttattgttctcctcctaacattgaattccaacagtagaactcagcaatatattccgagttccatctccgctgcatttgattttacctcaggatctatatcccctgaacaacaagtcatctctgaggaaaatgatgctaaaaaattagagcaaagtgctctgaattcagaggcaagcgaagactccgaagccgtacatcgagcttttttattttcccatcccagagaagaaggagccgtgcctccagggtgaggcagagagcaaggcctctaagctctttggcaactactcccgggatcagcccatcttcctgcggcttgaggattatttctgggtcaagacgccatctgcttatgagctgccctatgggaccaaggggagtgaggatctgctcctccgggtgctagccatcaccagctcctccatccccaagaacatccagagcctcaggtgccgccgctgtgtggtcgtggggaacgggcaccggctgcggaacagctcactgggagatgccatcaacaagtatgatgtggtcatcagattgaacaatgccccagtggctggctatgagggtgacgtgggctccaagaccaccatgcgtctcttctaccctgaatctgcccacttcgaccccaaagtagaaaacaacccagacacactcctcgtcctggtagctttcaaggcaatggacttccactggattgagaccatcctgagtgataagaagcgggtgcgaaagggtttctggaaacagcctcccctcatctgggatgtcaatcctaaacagattcggattctcaaccccttcttcatggagattgcagctgacaaactgctgagcctgccaatgcaacagccacggaagattaagcagaagcccaccacgggcctgttggccatcacgctggccctccacctctgtgacttggtgcacattgccggctttggctacccagacgcctacaacaagaagcagaccattcactactatgagcagatcacgctcaagtccatggcggggtcaggccataatgtctcccaagaggccctggccattaagcggatgctggagatgggagctatcaagaacctcacgtccttctaagaattcgacacttctaaataagcggatctcctatgccttcacgatttatagtttccattatcaagtatgcctatattagtatatagcatctttagatgacagtgttcgaagtttcacgaataaaagataatattctactttttgctcccaccgcgtttgctagcacgagtgaacaccatccctcgcctgtgagttgtacccattcctctaaactgtagacatggtagcttcagcagtgttcgttatgtacggcatcctccaacaaacagtcggttatagtttgtcctgctcctctgaatcgtctccctcgatatttctcattttccttcgcatgccctcgagggccgactcggccggccggcc

SEQ ID NO: 22 - DNA, 17 bp, Artificial Sequence
  Primer CA027
ggaaagacgggtgcaac

SEQ ID NO: 23 - DNA, 23 bp, Artificial Sequence
  Primer CA028
cccaacgtcatataatgatctga

SEQ ID NO: 24 - DNA, 22 bp, Artificial Sequence
  Primer CA017
atgttcgccaacctaaaatacg

SEQ ID NO: 25 - DNA, 20 bp, Artificial Sequence
  Primer CA018
ttacaaggatggctccaagg

SEQ ID NO: 26 - DNA, 18 bp, Artificial Sequence
  Primer CA046
tccagggctactacaaga

SEQ ID NO: 27 - DNA, 17 bp, Artificial Sequence
  Primer CR008
ccagctccttccggtca

SEQ ID NO: 28 - DNA, 20 bp, Artificial
  Primer CA040
tggagaagataattggagat

SEQ ID NO: 29 - DNA, 19 bp, Artificial Sequence
  Primer CA041
gcggtcttagggaaacata

SEQ ID NO: 30 - DNA, 18 bp, Artificial Sequence
  Primer CD030
cccgaatacctcagactg

SEQ ID NO: 31 - DNA, 20 bp, Artificial Sequence
  Primer CD031
actcgatcagcttctgatag

SEQ ID NO: 32 - DNA, 19 bp, Artificial
  Primer CA095
cagtagctttaggcggttc

SEQ ID NO: 33 - DNA, 19 bp, Artificial
  Primer CA096
gctacgacagatgcaaagg

SEQ ID NO: 34 - DNA, 20 bp, Artificial
  Primer CB125
tggcgggttaattgcagaag

SEQ ID NO: 35 - DNA, 20 bp, Artificial Sequence
  Primer CB126
agtggatgatgctccattgg

SEQ ID NO: 36 - DNA, 20 bp, Artificial Sequence
  Primer CB144
aggaactggcgaagttgagt

SEQ ID NO: 37 - DNA, 20 bp, Artificial Sequence
  Primer CB145
actcctgcaaatccagagca

SEQ ID NO: 38 - DNA, 20 bp, Artificial Sequence
  Primer CB127
gcttgaggattatttctggg

SEQ ID NO: 39 - DNA, 19 bp, Artificial Sequence
  Primer CB104
tcagaaggacgtgaggttc